SEQUENCE LISTING (20 sequences)

SEQ ID NO: 1 - protein, 86 aa, Bos taurus
  VARIANT 3: note = V
  VARIANT 22: note = L
  VARIANT 34: note = M
  VARIANT 37: note = L
  VARIANT 45: note = K
  VARIANT 46: note = V
  VARIANT 48: note = I
  VARIANT 62: note = F
  VARIANT 64: note = V
  VARIANT 67: note = K
  VARIANT 69: note = H
  VARIANT 70: note = K
  VARIANT 82: note = F
  VARIANT 83: note = K
NQPGPPGPPGPPGSAGEPGPGGRPGFPGTPGMQGPQGERGLPGEXGERGLPGPPGPQGESRTGPPGSTGSRGPPGPPGRPGDSGIR

SEQ ID NO: 2 - protein, 86 aa, Bos taurus; identical to SEQ ID NO: 1
  VARIANT 2: note = F
  VARIANT 4: note = H
  VARIANT 13: note = K
  VARIANT 15: note = V
  VARIANT 16: note = K
  VARIANT 22: note = L
  VARIANT 35: note = M
  VARIANT 39: note = V
  VARIANT 40: note = V
  VARIANT 45: note = K
  VARIANT 67: note = K
  VARIANT 68: note = F
  VARIANT 69: note = I
  VARIANT 71: note = L

SEQ ID NO: 3 - protein, 86 aa, Bos taurus; identical to SEQ ID NO: 1
  VARIANT 2: note = K
  VARIANT 15: note = K
  VARIANT 16: note = I
  VARIANT 22: note = M
  VARIANT 33: note = K
  VARIANT 34: note = L
  VARIANT 36: note = H
  VARIANT 37: note = L
  VARIANT 39: note = V
  VARIANT 45: note = K
  VARIANT 46: note = V
  VARIANT 48: note = V
  VARIANT 68: note = L
  VARIANT 72: note = F

SEQ ID NO: 4 - protein, 86 aa, Bos taurus; identical to SEQ ID NO: 1
  VARIANT 2: note = K
  VARIANT 16: note = V
  VARIANT 17: note = K
  VARIANT 21: note = L
  VARIANT 37: note = V
  VARIANT 45: note = K
  VARIANT 46: note = I
  VARIANT 49: note = V
  VARIANT 61: note = M
  VARIANT 68: note = F
  VARIANT 70: note = H
  VARIANT 73: note = L
  VARIANT 82: note = F
  VARIANT 84: note = K

SEQ ID NO: 5 - protein, 86 aa, Bos taurus; identical to SEQ ID NO: 1
  VARIANT 2: note = K
  VARIANT 5: note = K
  VARIANT 15: note = V
  VARIANT 22: note = I
  VARIANT 34: note = H
  VARIANT 37: note = V
  VARIANT 38: note = K
  VARIANT 40: note = M
  VARIANT 44: note = F
  VARIANT 45: note = K
  VARIANT 46: note = V
  VARIANT 67: note = L
  VARIANT 68: note = K
  VARIANT 69: note = L

SEQ ID NO: 6 - protein, 86 aa, Bos taurus; identical to SEQ ID NO: 1
  VARIANT 2: note = F
  VARIANT 4: note = L
  VARIANT 14: note = K
  VARIANT 29: note = L
  VARIANT 36: note = M
  VARIANT 45: note = K
  VARIANT 47: note = L
  VARIANT 49: note = V
  VARIANT 61: note = I
  VARIANT 67: note = V
  VARIANT 69: note = V
  VARIANT 73: note = H
  VARIANT 83: note = K
  VARIANT 84: note = K

SEQ ID NO: 7 - protein, 86 aa, Bos taurus; identical to SEQ ID NO: 1
  VARIANT 2: note = K
  VARIANT 16: note = F
  VARIANT 29: note = V
  VARIANT 40: note = K
  VARIANT 44: note = L
  VARIANT 45: note = M
  VARIANT 63: note = I
  VARIANT 70: note = H
  VARIANT 72: note = V
  VARIANT 82: note = K
  VARIANT 83: note = L
  VARIANT 84: note = V

SEQ ID NO: 8 - protein, 86 aa, Bos taurus; identical to SEQ ID NO: 1
  VARIANT 5: note = V
  VARIANT 15: note = M
  VARIANT 16: note = L
  VARIANT 17: note = K
  VARIANT 21: note = K
  VARIANT 23: note = M
  VARIANT 29: note = H
  VARIANT 32: note = K
  VARIANT 39: note = F
  VARIANT 44: note = L
  VARIANT 45: note = K
  VARIANT 46: note = V
  VARIANT 64: note = V
  VARIANT 70: note = I
  VARIANT 73: note = K

SEQ ID NO: 9 - protein, 86 aa, Bos taurus; identical to SEQ ID NO: 1
  VARIANT 15: note = K
  VARIANT 23: note = V
  VARIANT 34: note = M
  VARIANT 45: note = K
  VARIANT 46: note = V
  VARIANT 47: note = K
  VARIANT 48: note = L
  VARIANT 49: note = H
  VARIANT 62: note = F
  VARIANT 72: note = I
  VARIANT 83: note = K
  VARIANT 84: note = V
  VARIANT 86: note = L

SEQ ID NO: 10 - protein, 86 aa, Bos taurus; identical to SEQ ID NO: 1
  VARIANT 14: note = K
  VARIANT 17: note = V
  VARIANT 22: note = V
  VARIANT 29: note = K
  VARIANT 34: note = M
  VARIANT 35: note = I
  VARIANT 44: note = L
  VARIANT 45: note = K
  VARIANT 47: note = L
  VARIANT 48: note = V
  VARIANT 64: note = H
  VARIANT 83: note = F

SEQ ID NO: 11 - protein, 86 aa, Bos taurus; identical to SEQ ID NO: 1
  VARIANT 2: note = K
  VARIANT 3: note = V
  VARIANT 15: note = F
  VARIANT 38: note = V
  VARIANT 44: note = M
  VARIANT 45: note = K
  VARIANT 49: note = L
  VARIANT 63: note = I
  VARIANT 64: note = K
  VARIANT 68: note = K
  VARIANT 69: note = H
  VARIANT 82: note = V
  VARIANT 84: note = L

SEQ ID NO: 12 - protein, 86 aa, Bos taurus; identical to SEQ ID NO: 1
  VARIANT 1: note = M
  VARIANT 2: note = F
  VARIANT 15: note = K
  VARIANT 17: note = H
  VARIANT 22: note = L
  VARIANT 35: note = V
  VARIANT 37: note = K
  VARIANT 39: note = V
  VARIANT 45: note = K
  VARIANT 47: note = V
  VARIANT 68: note = K
  VARIANT 73: note = I
  VARIANT 83: note = L

SEQ ID NO: 13 - protein, 86 aa, Bos taurus; identical to SEQ ID NO: 1
  VARIANT 1: note = H
  VARIANT 14: note = L
  VARIANT 17: note = K
  VARIANT 21: note = V
  VARIANT 23: note = K
  VARIANT 37: note = V
  VARIANT 40: note = M
  VARIANT 45: note = V
  VARIANT 47: note = L
  VARIANT 61: note = K
  VARIANT 62: note = F
  VARIANT 69: note = I

SEQ ID NO: 14 - protein, 86 aa, Bos taurus; identical to SEQ ID NO: 1
  VARIANT 15: note = L
  VARIANT 16: note = V
  VARIANT 17: note = L
  VARIANT 37: note = K
  VARIANT 39: note = V
  VARIANT 45: note = K
  VARIANT 48: note = K
  VARIANT 49: note = V
  VARIANT 62: note = I
  VARIANT 64: note = M
  VARIANT 71: note = H
  VARIANT 73: note = F

SEQ ID NO: 15 - protein, 86 aa, Bos taurus; identical to SEQ ID NO: 1
  VARIANT 14: note = K
  VARIANT 36: note = K
  VARIANT 37: note = F
  VARIANT 45: note = K
  VARIANT 46: note = K
  VARIANT 49: note = V
  VARIANT 61: note = V
  VARIANT 64: note = I
  VARIANT 68: note = L
  VARIANT 72: note = L
  VARIANT 82: note = H
  VARIANT 83: note = V
  VARIANT 86: note = M

SEQ ID NO: 16 - protein, 86 aa, Bos taurus; identical to SEQ ID NO: 1
  VARIANT 2: note = H
  VARIANT 3: note = K
  VARIANT 13: note = V
  VARIANT 16: note = M
  VARIANT 17: note = L
  VARIANT 21: note = F
  VARIANT 23: note = L
  VARIANT 33: note = K
  VARIANT 35: note = I
  VARIANT 45: note = K
  VARIANT 48: note = V
  VARIANT 62: note = L
  VARIANT 73: note = V
  VARIANT 86: note = K

SEQ ID NO: 17 - protein, 86 aa, Bos taurus; identical to SEQ ID NO: 1
  VARIANT 2: note = V
  VARIANT 16: note = K
  VARIANT 21: note = V
  VARIANT 32: note = V
  VARIANT 34: note = H
  VARIANT 39: note = L
  VARIANT 44: note = K
  VARIANT 45: note = M
  VARIANT 46: note = K
  VARIANT 49: note = I
  VARIANT 68: note = I
  VARIANT 71: note = L
  VARIANT 72: note = M
  VARIANT 73: note = F

SEQ ID NO: 18 - protein, 86 aa, Bos taurus; identical to SEQ ID NO: 1
  VARIANT 4: note = L
  VARIANT 15: note = K
  VARIANT 16: note = V
  VARIANT 29: note = V
  VARIANT 36: note = K
  VARIANT 44: note = H
  VARIANT 45: note = K
  VARIANT 46: note = V
  VARIANT 48: note = L
  VARIANT 64: note = F
  VARIANT 69: note = K
  VARIANT 72: note = I
  VARIANT 83: note = M

SEQ ID NO: 19 - protein, 86 aa, Bos taurus; identical to SEQ ID NO: 1
  VARIANT 1: note = L
  VARIANT 2: note = V
  VARIANT 3: note = V
  VARIANT 5: note = M
  VARIANT 16: note = K
  VARIANT 22: note = K
  VARIANT 23: note = V
  VARIANT 38: note = I
  VARIANT 39: note = F
  VARIANT 40: note = L
  VARIANT 45: note = K
  VARIANT 46: note = H
  VARIANT 68: note = F
  VARIANT 70: note = K

SEQ ID NO: 20 - protein, 86 aa, Bos taurus; identical to SEQ ID NO: 1
  VARIANT 3: note = M
  VARIANT 16: note = H
  VARIANT 21: note = K
  VARIANT 22: note = L
  VARIANT 29: note = K
  VARIANT 38: note = L
  VARIANT 44: note = K
  VARIANT 45: note = K
  VARIANT 46: note = K
  VARIANT 49: note = V
  VARIANT 61: note = V
  VARIANT 67: note = F
  VARIANT 84: note = I
  VARIANT 86: note = V

(19) United States
(12) Patent Application Publication
(10) Pub. No.: US 2025/0032592 A1
(43) Pub. Date: Jan. 30, 2025

(54) Optimised Protein Comprising the Amino Acids Essential for Human Nutrition

(71) Applicant: UNIVERSIDAD NACIONAL AUTONOMA DE MEXICO, Ciudad de Mexico (MX)

(72) Inventors: Gabriel Del Rio Guerra, Morelos (MX); Sofia Natali Mendoz Cabrera, Ciudad de Mexico (MX)

(21) Appl. No.: 18/710,696

(22) PCT Filed: Oct. 27, 2022
(86) PCT No.: PCT/MX2022/050108

§ 371 Date: May 16, 2024

(30) Foreign Application Priority Data
Nov. 19, 2021 (MX) MX/A/2021/014268

(51) Int. Cl. A61K 38/39; C07K 14/78; C12P 21/02

(52) U.S. Cl. CPC A61K 38/39; C07K 14/78; C12P 21/02

(57) ABSTRACT

The present invention relates to an optimized protein comprising the amino acid sequences as set forth in SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; SEQ ID NO. 14; SEQ ID NO. 15; SEQ ID NO. 16; SEQ ID NO. 17; SEQ ID NO. 18; SEQ ID NO. 19; SEQ ID NO. 20, wherein said optimized protein comprises all essential amino acids in ratios suitable for human nutrition.

### FIELD OF THE INVENTION

The present invention is related to the techniques and principles used in Biochemistry for the study and investigation of cells, as well as the chemical nature of these, in addition to the development of new chemical processes in the biological systems of both cells and tissues and organs, and more particularly, it is related to an optimized protein comprising the essential amino acids in the ratios suitable for human nutrition.

### BACKGROUND OF THE INVENTION

Deterioration of muscle function and muscle mass, also known as sarcopenia, is a natural phenomenon that occurs in humans after the age of 40. Every 10 years a human loses on average 8% of muscle mass, so that by the age of 65, people will have lost one-fifth of their muscle mass and thus will have considerably lost the capacity for autonomous movement. Sarcopenia not only limits the capacity for autonomous movement, but is also a promoter of the development of metabolic syndrome and with it diabetes, propensity to develop cancer and increased mortality, among others.

Sarcopenia can be delayed by ingesting large amounts of protein or essential amino acids. However, another natural phenomenon that occurs during aging is the loss of appetite, which makes it difficult for these treatments to give the desired results.

There are several problems associated with natural protein sources, namely:
1) they are very diluted (for example, out of every 100 grams of beef, 30 grams are protein);
2) they do not have the amounts of essential amino acids necessary for the human diet;
3) amino acids are not readily available, for example, less than 50% of the amino acids present in plant proteins are available;
4) they require large quantities of potable water for their production, for example, to produce 1 kg of beef requires 16 thousand liters of water, 1 kg of chocolate requires 19 thousand liters of water, among others.

Amino acids are indispensable for life and their consumption is essential, not only as an energy source, but also for the oxygenation of the organism, the proper functioning of the immune system and tissue repair (National Research Council (2001) “Protein and Amino Acids”).

Amino acids have been classified into essential and non-essential amino acids by animal growth monitoring and nitrogen balance assays. Essential amino acids are those whose carbon skeletons cannot be synthesized de novo by the organism and therefore must be provided in the diet (Wu et al., 2009; Wu et al., 2014). Of the twenty (20) essential amino acids (EAA) required to build proteins, nine (9) of them are considered essential, namely: histidine (His), isoleucine (Ile), leucine (Leu), lysine (Lys), methionine (satisfied), phenylalanine (Phe), threonine (Thr), tryptophan (Trp) and valine (Val).

On the other hand, non-essential amino acids (NEAA) are also required for protein synthesis, but are synthesized by tissues from other essential and non-essential amino acids, and are the following: alanine (Ala), arginine (Arg), asparagine (Asn), aspartate (Asp), glutamate (Glu), glutamine (Gln), glycine (Gly), proline (Pro) and serine (Ser) for adult, non-carnivorous mammals (National Research Council (2001), “Protein and Amino Acids”). In turn, these NEAA have been classified as conditionally essential because their rates of use are higher than the rates of synthesis under certain conditions such as pregnancy, wounds, infections, etc. These are glutamate, glycine, glutamine, proline and taurine in mammals (National Research Council (2001) “Protein and Amino Acids). Even though elevated levels of glutamine, glutamate and aspartate have been found to have a neurotoxic effect (Wu et al., 2014). Cysteine and tyrosine that are not synthesized by the body are not considered as essential, because these can be formed from methionine and phenylalanine in the liver, respectively (Wu., 2013). If the AA are not present in adequate ratios, protein synthesis is decreased and protein breakdown is increased (Brunton et al., 1998; Zello et al., 1995; Duffy et al., 1981).

EAA are primarily responsible for regulating muscle protein synthesis, with leucine being the EAA that plays the most important role in this process (Volpi et al., 2003; Garlick et al., 2005). Katsanos et al., 2006, reported that leucine has a unique role in stimulating muscle protein synthesis in older adults.

Protein consumption is fundamental given its importance in transport, structural, regulatory, contractile, immunological, catalytic and energetic functions. The nutritional value of proteins has been measured by parameters, such as caloric content or weight, but this does not provide any information on the content of essential amino acids (EAA) in the proteins consumed by humans (Tessari et al., 2016). The most important aspect of proteins, from a nutritional point of view, is their amino acid composition.

The quality of a protein is mainly determined by its digestibility and bioavailability, as well as by its content of EAA (FAO/WHO, 1991), which cannot be synthesized by the organism and, therefore, must be consumed in the diet to ensure the synthesis of the proteins required by the human body. The World Health Organization (WHO) establishes the optimal ratios of EAA required in the human daily intake (RDA).

The intake of quality proteins with EAA that the body cannot synthesize is of utmost importance because these are the key substrates for preserving or gaining muscle mass and for ensuring the synthesis of proteins required by the body (Millward et al., 2008). Identifying alternative protein sources that are affordable and of high nutritional value, whose production is sustainable, has become a very relevant issue due to the increasing global demand for food (Hussein et al., 2017).

New technologies are needed to find alternative protein supplements to replace traditional sources such as soybeans (Seo et al., 2008). The availability of soil for soybean crops is limited and anti-nutritional factors such as trypsin inhibitors, lectin and tannins, present in legumes such as soybeans, have been reported to increase protein losses by inducing intestinal paralysis in animal models, which would produce less protein hydrolysis and thus a reduced absorption of AA (Salgado et al., 2002).

In the 1990's the concept of the ideal protein became popular among nutritionists at the University of Illinois (Stein et al., 1994). However, the idea of a perfect balance of AA has been discussed since 1946 (Mitchell and Block, 1946) and to date no protein has been reported that contains EAA in the recommended ratio for human daily intake.

Current protein production systems are based on the generation of large quantities of proteins, which require large amounts of soil use and result in the emission of high amounts of greenhouse gases, but not in the production of high quality proteins (Tessari et al., 2016). Tessari et al. reevaluated the environmental footprint by taking into account a key factor in the quality of proteins consumed by humans, namely the EAA content. Soil use was recalculated for the production of 13 g of EAA or to ensure the RDA of each EAA. This study concludes that production of quality plant proteins in sufficient quantities to satisfy the EAA RDA would require increased soil use and higher greenhouse gas emissions, while the soil use for beef and soybeans is not changed as much because they are closer to the EAA content.

The FAO (Food and Agriculture Organization of the United Nations) estimates indicate that 70% more food will have to be produced by 2050, challenging the global capacity to provide enough food (Veldkamp et al., 2015). A 2018 investigation evaluated food production systems regarding soil use and greenhouse gas emissions if the diet recommended by Harvard nutritionists (HHEP: Harvard Healthy Eating Plate) (Bahadur et al., 2018). Estimates show that current agricultural systems overproduce grains, fats and sugars, but do not produce enough protein to satisfy the nutritional needs of the current population and the expected population by 2050, which will increase from 7 to 9.8 billion people.

At present, there is no report of a natural or synthetic protein with the recommended ratio of EAA. A protein with this composition would not only have a nutritional impact, but could also result in more affordable costs and less soil use than current production systems.

In the prior art, several documents were found that are related to the subject matter of the present invention, such is the case of international patent application No. PCT/US2013/071091 (International Publication No. WO 2014/081884), which proposes to genetically modify proteins to change their amino acid composition for health purposes; it also presents the expression of some selected proteins; in particular, example 14 discusses the production of the protein mostly secreted by Aspergillus niger (PDB: 3EQA, glucoamylase catalytic domain) that was mutated in its loops with more than 4 amino acids in length to include essential amino acids (F, L, I, M, V, T, K, R, W); these mutations increased by 41, 44 and 6% the content of essential amino acids regarding the original protein. The example of a protein secreted by Bacillus that is enriched in essential amino acids (H, I, L, K and M) is also given. It recognizes the low content of essential amino acids in natural sources and the various problems associated with the intake of natural proteins in the human diet. It is proposed that it is possible to design polypeptides with the required essential amino acid composition, but it recognizes the technical difficulty in achieving this. To reduce the risk of failure in constructing a protein with all essential amino acids, they focus on designing proteins that supplement one or more amino acids that are necessary in the diet of the human to be fed using conservative substitutions (1: serine, threonine; 2: aspartic acid, glutamic acid; 3: asparagine, glutamine; 4: arginine, lysine; 5: isoleucine, leucine, methionine, alanine, valine; and 6: phenylalanine, tyrosine, tryptophan); specifically design proteins that have up to 5 times the ratio of essential amino acids (histidine, isoleucine, leucine, lysine, methionine, phenylalanine, threonine, tryptophan, and valine); or aromatic (phenylalanine, tryptophan, tyrosine, histidine and thyroxine); or branched (leucine, isoleucine and valine); or RQL (arginine, glutamine and leucine) based on multiple alignments; substitutions were guided based on the frequency of occurrence of amino acids at each position. Mutation selection was based on the estimation of 6 factors: amino acid probability (AALike), amino acid type probability (AATLike), position entropy (Spos), entropy per amino acid and position (SAATpos), relative free energy of folding (ΔΔΔGfold), and secondary structure identity (LoopID). They also describe polypeptides that are designed to be free of certain amino acids (leucine, isoleucine, valine, arginine, histidine, lysine). The designed polypeptides possess 20 amino acids in length or more and are designed to increase the amount of the selected amino acid(s). The designed proteins resemble the initial protein in 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% in their amino acid sequences. These proteins are naturally secreted in the following microorganisms: Escherichia coli, Bacillus subtilis, Saccharomyces cerevisiae, Pichia pastoris, Corynebacterium, Synechocystis, and Synechococcus. However, the invention described in publication No. 081884 starts from a design that only allows increasing or eliminating essential amino acids, but does not allow a modification to achieve a particular composition, as is allowed in the present invention. Furthermore, said invention of publication No. 081884 describes proteins selected from databases for its designs, but none of said proteins are used in the present invention.

On the other hand, International Patent Application No. PCT/US1998/006673 (International Publication No. WO 1998/045458) relates to the development of seeds and seed storage proteins that are improved in the amount of amino acids that are essential for humans and animals. More specifically, this invention relates to the genetic engineering of Brazil Nut 2S albumin seed storage protein to contain a higher percentage of essential amino acid residues. Expression of a gene encoding this modified seed storage protein in transgenic plants results in increased accumulation of essential amino acids in the seeds of these plants. The production in plant (soybean) seeds of proteins (albumin) with a high content of tryptophan, cysteine and methionine is described. This is on the grounds that these amino acids are essential to the human diet and plants have few of them.

International Patent Application No. PCT/US1997/020441 (International Publication No. WO 1998/020133), provides polypeptides comprising protease inhibitors with increased amounts of essential amino acids and nucleotides encoding these peptides. It also provides transformed plants and seeds with improved nutritional value due to the expression of modified polypeptides. The production of proteins that function as protease inhibitors with increased essential amino acid content (K, W, M, T) and their expression in plants to increase their nutritional value is described. The design was based on conservative substitutions.

It is important to note that, in the prior art, various patent documents were found that refer to the use of essential amino acids, but none of them discuss or describe an invention that produces proteins with the balanced content of essential amino acids for the human diet, which is an important aspect of the invention described in the present patent application.

On the other hand, genetic engineering has been used to modify plants (for example, corn) that have an increase in the amount of the amino acid lysine that they produce (https://www.ncbi.nlm.nih.gov/pmc/articles/PMC2442549/). However, this approach does not solve the problems of excessive water use required for its production, it only provides one of the 11 essential amino acids and it is produced in a plant whose proteins are very diluted and not very available to humans.

Currently, the intake of free essential amino acids is used to address the problem of sarcopenia, and more particularly leucine, which is one of the twenty amino acids used by cells to synthesize proteins (https://www.ncbi.nlm.nih.gov/pmc/articles/PMC3183816/). However, the consequence of long-term free amino acid intake on health has not yet been evaluated. Animal studies show that the intake of free amino acids is not as efficient in animal nutrition (https://www.frontiersin.org/articles/10.3389/fped.2019.00563/full) and that it can generate colitis and inflammation (https://pubmed.ncbi.nlm.nih.gov/29209321/).

The risk to both human and animal health posed by the ingestion of free amino acids is demonstrated in a study conducted by the FDA in 1994, in which it was reported that the lack of regulation for the sale of free amino acids led to the death of people due to the presence of contaminants in these preparations (https://www.ncbi.nlm.nih.gov/books/NBK209070/).

Similarly, recent work shows that long-term sustained intake of free amino acids negatively affects health and life expectancy in animals (https://www.siencedirect.com/Science/Article/pii/S2468501119300082); (https://www.nature.com/articles/s42255-019-0059-2). In this sense, studies have already been initiated to evaluate whether the same occurs in humans.

Particularly, the invention described in international application PCT/US2013/071091, which describes the protein selected to improve the essential amino acid composition in A. niger, glucoamylase (470 AA), which possesses the following ratio of essential amino acids:
CS[C,M] Expected: 0.0105, CS[C,M] Observed: 0.01950912344241975, Ratio: 1.8580117564209284
CS[F,Y] Expected: 0.0175, CS[F,Y] Observed: 0.12041872570381736, Ratio: 6.881070040218134
CS[H] Expected: 0.007, CS[H] Observed: 0.010872084792543642, Ratio: 1.5531549703633774
CS[I] Expected: 0.014, CS[I] Observed: 0.04036903352408676, Ratio: 2.8835023945776257
CS[K] Expected: 0.021, CS[K] Observed: 0.020322422430736932, Ratio: 0.9677344014636634
CS[L] Expected: 0.0273, CS[L] Observed: 0.08298079113284501, Ratio: 3.0395894187855315
CS[T] Expected: 0.0105, CS[T] Observed: 0.07814890209095882, Ratio: 7.442752580091315
CS[V] Expected: 0.0182, CS[V] Observed: 0.060906466077756176, Ratio: 3.3465091251514383
CS[W] Expected: 0.0028, CS[W] Observed: 0.055358833891450694, Ratio: 19.771012104089532.

That is to say, while there are essential amino acids that are present in the recommended ratio (K, H), there are others that are in excess (W, T, F/Y) so that the invention was only concerned with increasing the amount of essential amino acids, but not with producing a protein that has a balanced ratio for the human diet. The other problem is that the selected protein has an enzymatic activity that may not be convenient to ingest, so the authors propose some strategies to inactivate it. One problem with mutating amino acids important for enzyme activity is that protein stability depends on these amino acids.

As can be seen from the above discussion, the inventions described in application No. PCT/US1998/006673 and No. PCT/US1997/020441 do not solve the problems of the excessive use of water required for its production and it is produced in vegetables whose proteins are very diluted and are poorly available to humans.

### SUMMARY OF THE INVENTION

The present invention relates to an optimized protein comprising essential amino acids in the ratios suitable for human nutrition, wherein said optimized protein was obtained using an algorithmic method, wherein from a selection in public databases of protein sequences, the sequence that came closest to containing the adequate ratio of essential amino acids for human nutrition was selected, considering also previous evidence of its expression in heterologous systems and its nature to be secreted, we proceeded to develop the algorithmic method that allowed making changes in the selected sequence so that it complies with the highest possible amount of essential amino acids in the adequate ratio for human nutrition.

For selection of the protein sequence that came closest to containing the appropriate ratio of essential amino acids, a search of public protein sequence databases was performed consisting of the following steps: 1) defining the ratio of essential amino acids per gram of total protein desired to be found in any protein (PAAE), wherein this ratio is specified in a range of values (RVPAAE); 2) defining how many essential amino acids must comply with the RVPAAE (AAEE: Expected Essential Amino Acids), which can be from 1 to 9; 3) searching in protein sequence databases, those that satisfy the steps 1) and 2); 4) repeating steps 1), 2) and 3) for different possible values of AAEE; 5) selecting proteins whose AAEE are higher, preferably those proteins that satisfy a higher number of essential amino acids in the right ratio for human nutrition.

This procedure can be performed to supplement the amino acids present in a desired food (for example, milk, egg, etc.) or to find a protein that alone can satisfy the requirements of essential amino acids for human nutrition.

From the sequence selected in the previous procedure, changes were made in that sequence to have it contain the essential amino acids in the desired ratios of essential amino acids, for which the optimization algorithm known as “simulated annealing” was used, wherein the steps of the optimization algorithm are as follows: a) starting the power of the system; b) calculating the value of the energy function of the sequence; c) printing the solution, provided that the value of the energy function of the sequence is equal to 0; otherwise continue with the following steps; d) randomly selecting the positions in the sequence to be mutated; the number of positions is the method parameter specified in the following step e); e) verifying that the selected positions are among the positions susceptible to be changed specified by the parameter of the corresponding method with in step b); f) randomly selecting the essential amino acids for which the amino acids present in the selected positions are to be changed; g) carrying out the substitutions specified in step e) on the positions selected in step d); if the new sequence reduces the energy value, keep it, otherwise evaluate whether to keep that sequence by applying the following formula:

$${e}^{( previous energy - new energy ) / temperature}$$

if the result gives a number greater than a randomly chosen number between 0 and 1, then the sequence is retained for the next cycle; and h) repeating the above steps until the number of sequences printed equals the method parameter specified in step g), or a value in the system energy less than 1 has been reached.

For obtaining the optimized protein of the present invention, from the universe of amino acid sequences found in the search, the following optimized amino acid sequences set forth in the attached list of sequences were selected in a preferable, but not limiting manner from said present invention: SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; SEQ ID NO. 14; SEQ ID NO. 15; SEQ ID NO. 16; SEQ ID NO. 17; SEQ ID NO. 18; SEQ ID NO. 19; SEQ ID NO. 20, which comprise the ratios indicated for each essential amino acid (Observed and Expected; Ratio indicates the ratio between Observed and Expected); the observed sequence identity with the template sequence is also indicated.

In additional aspect of the present invention, from among said preferred optimized amino acid sequences were selected most preferably the amino acid sequences with which experimental tests were carried out, SEQ ID NO. 12 and SEQ ID NO. 16.

In yet another further aspect of the present invention, an amino acid sequence of name SEQ ID NO. 16 to express it in yeast Pichia pastoris under 2 different promoters: pGAP and pAOX1.

The pGAPZ alpha plasmid was used to include the gene coding for the protein of SEQ ID NO. 16, using the codons of preferential use in the yeast Pichia pastoris. The corresponding nucleotide sequence for protein of SEQ ID NO. 16 is as follows:

5′-
CAACCACCCAGGTCCACCAGGTCCACCAGGTCCACCAGTTTCTGCTATG
TTGCCAGGTCCATTCGGTTTGCCAGGTTTCCCAGGTACTCCAGGTATGA
AGGGTATTCAAGGTGAGAGAGGTTTGCCAGGTGAGAAGGGTGAGGTTGG
TTTGCCAGGTCCACCAGGTCCACAAGGTGAGTCTAGATTGGGTCCACCA
GGTTCTACTGGTTCTAGAGGTGTTCCAGGTCCACCAGGTAGACCAGGTG
ACTCTGGTATTAAG-3′

Strains of the yeast Pichia pastoris X33 overexpressing the plasmid carrying the gene coding for the protein of SEQ ID NO. 16 were isolated using different concentrations of the antibiotic Zeocin (400 μg/mL, 800 μg/mL and 1200 μg/mL). Two strains were isolated that grew at the highest concentration of Zeocin expressing the protein gene of SEQ ID NO. 16 under the pAOX1 promoter and 3 strains that grew at the highest concentration of Zeocin expressing the protein gene of SEQ ID NO. 16 under the pGAP promoter.

As already discussed in the Background of the Invention, the nutritional quality of a protein is mainly given by its digestibility and EAA content, which cannot be synthesized by the organism and, therefore, it is necessary to consume them in the diet to ensure the synthesis of the proteins required by the human body. The WHO establishes the optimal ratios of EAA required in the human daily intake (RDA).

### OBJECTS OF THE INVENTION

Taking into account the problems encountered in the prior art, it is an object of the present invention to provide an optimized protein that includes the essential amino acids in the ratios suitable for human nutrition.

It is a further object of the present invention to provide optimized protein having the nutritional composition of essential amino acids (EAA) required in the human diet by bioinformatic analysis of existing databases and which has been previously reported by WHO.

Another object of the present invention is to provide optimized protein that complements the EAA content of natural protein sources such as egg, milk, beef, chicken, pork and fish.

It is still further an object of the present invention to generate variants of the optimized protein that include the EAA required for human nutrition.

It remains a further object of the present invention to clone the gene of the protein closest to the RDA into an expression vector.

The foregoing and other objects and characteristics and advantages of the present invention will become more obvious by describing in greater detail the embodiments of said present invention and by referring to the accompanying drawings, wherein the latter, in addition to forming part of the present invention, provide additional insight into said embodiments, but do not constitute a limitation of the present invention. In the drawings, the same numerical references generally represent the same or similar parts or steps.

### BRIEF DESCRIPTION OF THE FIGURES

The novel aspects considered to be characteristic of the present invention will be set forth with particularity in the appended claims. However, the invention itself, both by its organization, as well as by its method of operation, in conjunction with other objects and advantages thereof, will be better understood in the following detailed description of the modes of the present invention, when read in connection with the accompanying drawings, in which:

FIG. 1 is a graph showing the number of proteins in which the ratio of each of the 9 essential amino acids (EAA) is satisfied, wherein the 5 proteins analyzed were the following: synaphin, uncharacterized protein, vasotocin, collagen type XII and regulatory protein RECX_PSEMY.

FIG. 2 is a graph showing the ratios of each of the 9 EAA in the 5 proteins found, wherein the ratio (1×-13×) of each EAA (T, M, F, K, H, V, I, L, W) in the uncharacterized protein from Macaca fascicularis (bars in pink color), synaphin from Doryteuthis pealeii (bars in blue color), collagen protein from Bos taurus (bars in yellow color), regulatory protein from Pseudomonas mendocina (bars in red color) and Vasotocin-neurophysin from Gallus gallus (bars in green color).

FIG. 3 is a graph showing the ratio of each AA in the Uniprot (SwissProt and TrEMBL) and PDB databases. The ratio of each AA is equal to the number of times the AA was found in the annotated proteins in the database*average mass of the AA/average mass of the annotated proteins.

FIG. 4 shows the length distribution of proteins annotated in SwissProt and PDB. Number of proteins in PDB (red dots) and SwissProt (blue dots) that fall within certain length ranges, which increase by 50 in 50 AA.

FIG. 5 shows the length distribution of proteins annotated in TrEMBL. Number of proteins (purple dots) in TrEMBL that fall within certain length ranges, which increase by 50 in 50 AA.

FIG. 6 shows the AA ratios of bacterial proteomes. The ratio of each AA is equal to the number of times the AA was found in the annotated proteins in the database*average mass of the AA/average mass of the annotated proteins. The 20 AA were taken into account.

FIG. 7 shows the AA ratios of fungal proteomes. The ratio of each AA is equal to the number of times the AA was found in the annotated proteins in the database*average mass of the AA/average mass of the annotated proteins.

FIG. 8 shows the AA ratios of plant proteomes. The ratio of each AA is equal to the number of times the AA was found in the annotated proteins in the database*average mass of the AA/average mass of the annotated proteins.

FIG. 9 shows the AA ratios of animal proteomes. The ratio of each AA is equal to the number of times the AA was found in the annotated proteins in the database*average mass of the AA/average mass of the annotated proteins.

FIG. 10 shows the AA ratios of virus proteomes. The ratio of each AA is equal to the number of times the AA was found in the annotated proteins in the database*average mass of the AA/average mass of the annotated proteins.

FIG. 11 shows the ratios of AA in the proteomes of animals, plants, fungi, viruses and bacteria. The ratio of each AA is equal to the number of times the AA was found in the annotated proteins in the database*average mass of the AA/average mass of the annotated proteins.

FIG. 12 shows a picture of an acrylamide gel stained with Coomasie blue staining proteins obtained in the supernatant of Pichia pastoris cells expressing the sequence SEQ ID NO. 16 under the promoter pGAP.

FIG. 13 shows a picture of an acrylamide gel stained with Coomasie blue staining proteins obtained in the supernatant of Pichia pastoris cells expressing the sequence SEQ ID NO. 16 under the pAOX1 promoter.

### DETAILED DESCRIPTION OF THE EMBODIMENTS OF THE INVENTION

To date, neither protein design nor protein engineering has been used to optimize the composition of essential amino acids for the human diet, but simply to improve the stability of proteins, change the activity of proteins for use in industrial processes, recognize molecules specifically, for example, in antibodies.

The lack of nutritional quality proteins to feed the population, the environmental impact of the current production systems of both vegetable and animal proteins, as well as the low content of essential amino acids (EAA) in vegetable proteins that implies consuming a great variety of vegetable protein sources to satisfy human requirements, were the reasons for which the present invention was carried out.

In view of the foregoing, the inventors of the present invention carried out two main activities, namely: i) a search for proteins of high nutritional value that do not depend on plant or animal systems; and ii) a proposal for a sustainable expression system of a protein for human consumption. Based on said two activities, in accordance with a particularly preferred embodiment of the present invention, an optimized protein comprising the essential amino acids in the ratios suitable for human nutrition was obtained, wherein said optimized protein was obtained using an algorithmic method developed by the inventors themselves of said present invention.

From a selection in public databases of protein sequences, the sequence that came closest to containing the adequate ratio of essential amino acids for human nutrition was selected, also considering previous evidence of its expression in heterologous systems and its nature to be secreted, we proceeded to develop the algorithmic method that allowed making changes in the selected sequence to comply with the highest possible amount of essential amino acids in the adequate ratio for human nutrition.

The optimization algorithm used is known as simulated annealing. The idea of this algorithm is to simulate what happens in metallurgical practice wherein metals are first heated and then cooled, and in doing so, these metals are purified of impurities. The algorithm starts at a high temperature and terminates when the function to be optimized has been satisfied, or a minimum temperature has been reached.

The steps of the optimization algorithm are as follows:
a) starting the power of the system;
b) calculating the value of the energy function of the sequence;
c) printing the solution, provided that the value of the energy function of the sequence is equal to 0; otherwise continue with the following steps;
d) randomly selecting the positions in the sequence to be mutated; the number of positions is the method parameter specified in the following step e);
e) verifying that the selected positions are among the positions susceptible to be changed specified by the parameter of the corresponding method with in step b);
f) randomly selecting the essential amino acids for which the amino acids present in the selected positions are to be changed;
g) carrying out the substitutions specified in step e) on the positions selected in step d); if the new sequence reduces the energy value, keep it, otherwise evaluate whether to keep that sequence by applying the following formula:

$${e}^{( previous energy - new energy ) / temperature}$$

if the result gives a number greater than a randomly chosen number between 0 and 1, then the sequence is retained for the next cycle; and
h) repeating the above steps until the number of sequences printed equals the method parameter specified in step g), or a value in the system energy less than 1 has been reached; and
i) synthesizing or producing the protein with the desired amino acid sequence(s).

In accordance with the foregoing, for obtaining the optimized protein of the particularly preferred embodiment of the present invention, a selection was made of the optimized sequences in its essential amino acid composition. It is important to note that, using the algorithmic method described above, it was found that the solutions matched only a range of values for the ratio from 1.9 to 3.1.

As mentioned previously, for obtaining the optimized protein of the present invention, from the universe of amino acid sequences found in the search the following optimized amino acid sequences set forth in the attached sequence list were selected in a preferable, but not limiting manner from said present invention: SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; SEQ ID NO. 14; SEQ ID NO. 15; SEQ ID NO. 16; SEQ ID NO. 17; SEQ ID NO. 18; SEQ ID NO. 19; SEQ ID NO. 20, which comprise the ratios indicated for each essential amino acid (Observed and Expected; Ratio indicates the ratio between Observed and Expected); the observed sequence identity with the template sequence is also indicated.

The letters in the sequences correspond to the 20 amino acids present in nature: A, Alanine; C, Cysteine; D, Aspartic acid; E, Glutamic acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; Y, Tyrosine.

Any of these preferred optimized amino acid sequences theoretically satisfies the condition of containing amino acids in the proper ratio for human nutrition. However, in a further aspect of the present invention, from among said preferred optimized amino acid sequences the amino acid sequences with which the experimental tests were carried out, were selected in a more preferred manner, the following considerations being taken into account:
i) the dispersion between the obtained ratios of each amino acid was not very large, whereby said most preferred sequences of the present invention are the amino acid sequences named SEQ ID NO. 12 and SEQ ID NO. 16, which presented the lowest dispersion; wherein both sequences maintain the three-dimensional structure of the template protein according to the predictor of Zhang's group named i-TASSER (https://zhanggroup.org/I-TASSER/). This prediction anticipates that these variants will have no folding problems when expressed in a cell.

In yet another further aspect of the present invention, an amino acid sequence of name SEQ ID NO. 16 to express it in yeast Pichia pastoris under 2 different promoters: pGAP and pAOX1; wherein pGAP is a system in which gene and protein expression is induced in the presence of glucose and pAOX1 in the presence of methanol.

In the following, a description is given of the methodological strategy that was followed to construct the DNA plasmids including the still more preferred amino acid sequence of the present invention SEQ ID NO. 16 under the pGAP and pAOX1 promoters.

### Plasmids and Strains Used to Validate Optimized Protein Design

The pGAPZ alpha plasmid was used to include the gene coding for the protein of SEQ ID NO. 16, using the codons of preferential use in the yeast Pichia pastoris. The corresponding nucleotide sequence for protein of SEQ ID NO. 16 is as follows:

5′-
CAACCACCCAGGTCCACCAGGTCCACCAGGTCCACCAGTTTCTGCTATG
TTGCCAGGTCCATTCGGTTTGCCAGGTTTCCCAGGTACTCCAGGTATGA
AGGGTATTCAAGGTGAGAGAGGTTTGCCAGGTGAGAAGGGTGAGGTTGG
TTTGCCAGGTCCACCAGGTCCACAAGGTGAGTCTAGATTGGGTCCACCA
GGTTCTACTGGTTCTAGAGGTGTTCCAGGTCCACCAGGTAGACCAGGTG
ACTCTGGTATTAAG-3′

The letters correspond to the 4 nucleotide bases present in DNA: A, Adenine; G, Guanine; T, Thymine; C, Cytosine.

Strains of the yeast Pichia pastoris X33 overexpressing the plasmid carrying the gene coding for the protein of SEQ ID NO. 16 were isolated using different concentrations of the antibiotic Zeocin (400 μg/mL, 800 μg/mL and 1200 μg/mL). Two strains were isolated that grew at the highest concentration of Zeocin expressing the protein gene of SEQ ID NO. 16 under the pAOX1 promoter and 3 strains that grew at the highest concentration of Zeocin expressing the protein gene of SEQ ID NO. 16 under the pGAP promoter.

As already discussed in the Background of the Invention, the nutritional quality of a protein is mainly given by its digestibility and EAA content, which cannot be synthesized by the organism and, therefore, it is necessary to consume them in the diet to ensure the synthesis of the proteins required by the human body. The WHO establishes the optimal ratios of EAA required in the human daily intake (RDA).

There is currently no report of a natural or synthetic protein with the recommended ratio of EAA. A protein with this composition would not only have a nutritional impact, but could also result in more affordable costs and less soil use than current production systems.

The present invention will be better understood from the following example, which is presented only for illustrative purposes, but not limiting, in such a way as to allow the full understanding of the embodiments of the present invention, without implying that there do not exist other embodiments not illustrated and which can be put into practice based on the detailed description previously made. It is important to point out that the data and experimental results obtained in the example described below are only intended to provide the necessary elements to carry out the invention, and therefore should not be considered as limiting the scope of the invention.

### EXAMPLES

### I. Example

Search for a Protein in Databases with the EAA Composition Required by Humans.

The Uniprot (96,757,994 sequences) and PDB (144,871 sequences) databases were consulted. The 454,976 protein sequences comprising the 16,233 proteomes reviewed (SwissProt), as well as the 96,303,018 protein sequences comprising the 166,576 proteomes not reviewed (TrEMBL) were downloaded from Uniprot in fasta format. The file with the unreviewed protein sequences (96,303,018) consisting of 54 Gb was fragmented into 223 files of 260 Mb each to subsequently analyze each file by using a code developed in Java, which allowed finding the proteins that satisfied the following criteria:
the ratio of EAA closest to the requirement in the human daily intake of EAA according to WHO;
the length of the protein was chosen taking into consideration that about 50% of the proteins annotated in the databases are in this length range and that at lengths greater than 260 AA, proteins closer to the requirements were not found, but the number of substitutions to be made increased greatly, which implied that these proteins had to be modified to a large extent. In addition, the generation of protein mixtures showed us that concatenation of 3 sequences (each up to 260 AA) no longer generated protein results close to the requirements due to dilution of the EAA content to longer lengths. For the above reasons, the search was limited to sequences of 1-260 AA.

Proteins with the lowest number of AA substitutions were selected taking into account a percentage of total protein change of up to 20% (calculated as: sum of substitutions to be made/total AA length of the protein).

The Java code consisted of 3 methods, namely:
1. getSeqs: allowed to read protein sequences in fasta format;
2. getMass: entered the average atomic mass of each AA according to those reported in: http://education.expasy.org/student_projects/isotopident/htdocs/aa-list.html.
3. seqContainsEnoughAAE: allowed to obtain the sequences that satisfy the daily requirement of each EAA. For this purpose, the values previously established by the WHO for each AA were introduced. For demonstration purposes, the calculations used in the code are shown for the case of lysines (K). However, each formula was applied with each of the 9 EAA to find those proteins that satisfied the recommended daily intake.

The first formula used to determine the content of lysines in protein (given in atomic mass) was the following:

$$Score K = ( k * 128.1741 Da ) / SeqMass )$$

wherein:
k represents the number of lysines found in the protein analyzed;
128.1741 Da is the average mass of lysine; and
SeqMass is the sum of the average atomic masses of the AA that conform the protein.

Subsequently, the required ratios of each EAA were obtained by dividing the WHO RDA values by 100 g of protein. When divided by 100 g, it was normalized regarding the gram of protein consumed. Taking as an example the RDA value for lysine, which is 2100 mg:

$$Ratio K = 2.1 g / 100 g of protein$$

Thus, the required ratio of lysine was 0.021. After having obtained the required ratios of the other EAA, another formula was implemented to the code to know the substitutions that had to be made in each protein to adjust its AA composition to the RDA:

$$Ak = ( EAA\_K * SeqMass / 128.1741 Da ) - k$$

wherein:
Ak represents the number of substitutions per lysine;
EAA_K is the required ratio of lysine (0.021);
SeqMass represents the average mass of the protein; and
128.1741 Da is the average mass of lysine.

The number of 39 lysines initially contained in the protein was subtracted from the previous term, resulting in the number of substitutions required in K to comply with the RDA.

Finally, the average mass of an AA for the protein in question was subtracted from each AA (this was obtained by dividing SeqMass by the number of AA in that protein), thus giving the variation in mass that should be used to adjust the mass of the new protein once the changes for lysines had been made:

$$Ak 2 = ( 128.1741 Da - massMProt ) * Ak$$

wherein:
Ak2 represents the number of substitutions to be made in lysine taking into account the average mass of the protein;
128.1741 Da is the average mass of lysine;
massMProt is the average mass of the protein divided by the number of AA in that protein; and
Ak represents the number of lysine substitutions.

The code also received certain arguments, which could be modified at the time of execution:
The first of these was a ratio range that could go from 0.9-1.2 (1×), 1.9-3.1 (2-3×) or 2.9-4.1 (3-4×). A 1× ratio implied that the code would search for proteins with the required ratio of each EAA; whereas a ratio of 2-3× implied searching for proteins with double or triple the required ratio of each EAA. Using a ratio range allowed the proteins found not to deviate too much from their established RDA value, that is, using a minimum of 0.9 implied that each of the 9 EAA could be 10% below its required 1× ratio, so that by applying a maximum range of 1.2 each EAA could be 20% above its required 1× ratio.
Secondly, there was the number of EAA that were desired to fall within the required ratio, which was abbreviated as the NAPR value (number of EAA in the required ratio), wherein this value could range from 1 to 9, corresponding to the number of EAA that were desired to fall within the ratio ranges.

For the execution of the code with the PDB and Uniprot databases, NAPR values ranging from 1-4 were used because when using higher values the code did not yield positive results in the range of 0.9-1.2. The code was also run using ranges of 1.9-3.1 and 2.9-4.1 with NAPR values of 8.

Search for Protein Mixtures with the Appropriate EAA Composition.

In addition to looking for a protein that satisfied the EAA requirements, protein blends were sought that had the recommended EAA ratios. For this purpose, the previously developed code was used and modified to concatenate different protein sequences generating 1:1 protein mixtures. The code was run against the Uniprot database, receiving as an argument whether protein mixtures with 2, 3 or more sequences were made. That is, if this parameter was set to be equal to 2, the code concatenated the sequences of protein 1 with the other sequences in the database and then the sequence of protein 2 with all the other sequences, analyzing whether the mixtures generated satisfied the EAA requirements. A minimum range of 0.9, a maximum range of 1.2, a NAPR value ranging from 4-9 and a maximum length of 260 AA were used for each protein in the mixture.

Search for Proteins that Complement Natural Protein Sources: Eggs, Milk, Beef, Fish, Pork and Chicken.

The Uniprot and PDB databases were consulted to search for proteins to complement the EAA content of natural protein sources such as egg, pork, milk, beef, fish and chicken with other code developed in Java. To do this, the ratios in which each EAA was found in these foods were first calculated. The ratio of each EAA was obtained by dividing the g in which each EAA was present by 100 g of the protein source. Taking beef as an example, 100 g of this food has 2,002 mg of lysine (WHO). Thus, the ratio of K in beef was 0.02002 (2.002 g/100 g).

The code sought proteins that would complement the ratios of the 9 EAA for each of the 5 foods. Following the example of beef, beef lacked 98 mg of K to satisfy its requirement. The code was looking for proteins that had these missing 98 mg and that would supplement their ratio (0.020022) to satisfy the required ratio of 0.02100. A range of 0.9-1.2, a NAPR value of 1-8 and a maximum protein length of 260 and 900 AA were used to run the code.

Ratios in which Each AA is Found in the Proteomes and in the Uniprot and PDB Databases.

Having seen that no protein in Uniprot and PDB had all 9 EAA in the ratios recommended by the WHO, the ratios in which the AA were found in the annotated proteins were analyzed to see if these ratios were higher or lower than those required for human daily intake.

The ratios of the 20 AA were calculated as follows:

$$ratio of each AA = number of times the AA was found in the annotated proteins in the database * average mass of the AA / total mass of the annotated proteins .$$

The code also made it possible to obtain the number of proteins falling within a certain AA length range. For this, an argument of 50 was used, which implied that the number of proteins that fell within that length range was displayed increasing by 50 AA. Using this code, the ratios of AA in the proteomes of the following organisms were analyzed: Acynonyx jubatus, Alligator mississippiensis, Bos taurus, Camelus dromedarius, Gadus morhua, Gallus gallus, Sus scrofa, Glycine max, Oryza sativa, Paenibacillus polimyxa, Rhodobacter spheroides, Schizophyllum commune, Tuber melanosporum, Ustilago maydis, Lactobacillus casei and Saccharomyces cerevisiae.

For the above, proteomes were downloaded from the website of the National Center for Biotechnology Information (https://www.ncbi.nlm.nih.gov/genome/).

Synthesis of the Gene Coding for the Protein with the EAA Composition Required in Human Daily Intake.

Bioinformatic analysis of the databases allowed finding the protein that was closest to the EAA requirement. This was collagen type XII protein from Bos taurus (Uniprot ID: P25508). The gene encoding for the protein was optimized according to the use of codons from Pichia pastoris and was synthesized de novo by Gene Universal Company (229A Lake Dr. Newark DE 19702) in the vectors: pGAPZα A (constitutive) and pAOX1 (inducible).

Generation of Collagen Protein Variants from Bos taurus to have the EAA Composition in the Appropriate Ratio.

The algorithmic method described above allows changes to be made to the selected sequences so that they comply with the highest possible amount of essential amino acids in the appropriate ratio for human nutrition, generating variants of the collagen protein from Bos taurus that incorporated the AA substitutions necessary for the 9 EAA to be in the recommended ratios. The algorithmic method received as arguments the positions of the protein wherein it could be mutated and the AA by which these positions could be mutated, excluding proline and glycine, which were not mutated to avoid folding problems because these residues confer the helical structure to the collagen protein. The program also received as an argument the number of simultaneous substitutions that would be made to evolve the protein in each mutation cycle, being able to mutate from 1 to 20 AA residues at the same time. The algorithmic method gave variant results mutating the 20 AA and using a range of 1.9-3.1. This sequence was included in the pGAPZ alpha plasmid under the pGAP or pAOX1 promoter and expressed in Pichia pastoris strain X33.

### II. Results

The proteins and protein pools that were closest to human EAA requirements at 1×, 2×, 3× and 4× ratios, as well as the proteins that best complemented the EAA content of the natural protein sources, are shown in tables below. The ratios in which each AA was found in the databases and in the proteomes analyzed are shown in graphs and finally a section is included with the experimental results of the cloning of the gene that codes for the protein that was selected as the closest to the requirements.

### Proteins Closest to the EAA Requirement in SwissProt.

Running the code developed in Java against the SwissProt database resulted in 4 proteins of maximum 260 AA having up to 4 EAA in the required 1× ratio of each EAA as reported by WHO (range 0.9-1.2; NAPR=3 and 4) (refer to Table 1). Four protein sequences that best approximated the human dietary EAA requirement were identified by running the code with NAPR values of 4 (proteins from Macaca fascicularis, Pseudomonas mendocina and Gallus gallus) and 3 (protein from Bos taurus).

TABLE 1
Protein sequences identified in the SwissProt database (454,976 sequences).

| | ID | | Length | No. of AA | Total protein change (# |
|---|---|---|---|---|---|
| Protein | Uniprot | Organism | (AA) | substitutions | of substitutions/length) |
| Vasotocin- | P24787 | Gallus gallus | 161 | 27 | 16.77% |
| neurophysin | | | | | |
| Regulatory | A4XWQ3 | Pseudomonas | 150 | 25 | 16.66% |
| protein | | mendocina | | | |
| RECX_PSEMY | | | | | |
| Uncharacterized | Q9GKT8 | Macaca | 77 | 12 | 15.58% |
| protein C16orf86 | | fascicularis | | | |
| homolog | | | | | |
| (Fragment) | | | | | |
| Collagen alpha- | P25508 | Bos taurus | 88 | 7 | 8.13% |
| 1 (XII) chain | | | | | |

As can be seen in Table 1, the collagen type XII protein from Bos taurus annotated in the SwissProt database best approximated the RDA, requiring 7 AA substitutions in an 86 AA protein, with a total percentage change to be made of 8.13%, followed by the protein from Macaca fascicularis with 15.58%, Pseudomonas mendocina with 16.66%, and finally the protein from Gallus gallus with 16.77%.

### Proteins Closest to EAA Requirement in PDB and TrEMBL.

The protein search in PDB yielded only 1 result of a protein that had a maximum length of 260 AA and had up to 4 EAA in the ratio 1× (range 0.9-1.2) (see Table 2), whereas with the unrevised Uniprot sequences (TrEMBL) 911 proteins were obtained that were closest to the human daily requirement of EAA. When running the code with NAPR values of 5-9 there was no result, which is interesting since this value indicates that the proteins in the databases analyzed (Uniprot and PDB) comply with a maximum of 4 EAA in the 1× ratio recommended for human daily intake, while the other 5 EAA are outside that ratio and, therefore, it is necessary to make certain substitutions to the proteins found to comply with the EAA content, because there is no protein in nature with the required ratio of EAA.

To see if the used length range of maximum 260 AA was interfering with the generation of results with NAPR values greater than 4, the code was rerun using as argument the search for proteins up to 1000 AA, obtaining that there were also no proteins in this length range that had NAPR values of 5-9.

TABLE 2
Protein sequence identified in the
PDB database (144,871 sequences).

| | | | No. of AA | Total protein |
|---|---|---|---|---|
| Protein | Organism | Length AA | substitutions | change |
| Synaphin A | Doryteuthis | 79 | 11 | 13.92% |
| | pealeii | | | |

The sequence in Table 2 was obtained through the code developed in Java using a NAPR value of 4 and a range of 0.9-1.2.

On the other hand, as can be seen in Table 2, a sequence was identified in the PDB database that most closely approximated the EAA requirement in the human diet. Such sequence identified in PDB corresponded to synaphin A from squid Doryteuthis pealeii with a total protein percentage change of 13.92%.

Choice of Protein with EAA Content in the Closest 1× Ratio (Range=0.9-1.2) to that Recommended for Human Consumption from the SwissProt, PDB and TrEMBL Databases.

The proteins found in TrEMBL were discarded because most of these proteins are uncharacterized, their structure is unknown, they have not been expressed and there is no experimental evidence that they are truly proteins or ORFs.

The sequences from the PDB and SwissProt databases that best approximated the RDA with a maximum percentage of total protein change of 20% were evaluated, since this implied that fewer protein substitutions had to be made later and that it was as close as possible to the EAA RDA.

Five proteins were found in which the number of AA substitutions to be made was minimal regarding the total length of the protein. The identified proteins correspond to the following organisms: Macaca fascicularis (12 substitutions, length=77 AA), Doryteuthis pealeii (11 substitutions, length=79 AA), Bos taurus (7 substitutions, length=86 AA), Pseudomonas mendocina (25 substitutions, length=150 AA) and Gallus gallus (27 substitutions, length=161 AA).

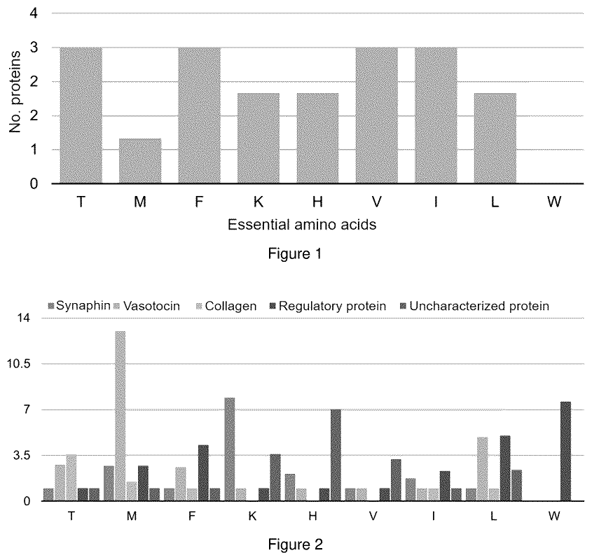

FIG. 1 of the accompanying drawings illustrates a graph showing the number of proteins in which the ratio of each of the 9 EAA is satisfied, wherein the 5 proteins analyzed were as follows: synaphin, uncharacterized protein, vasotocin, collagen type XII and regulatory protein RECX_PSEMY.

T, M, I and F EAA were in the required 1× ratio in the uncharacterized protein of Macaca fascicularis, T, V, L and F in the synaphin of Doryteuthis pealeii, I, L and F in collagen protein from Bos taurus, K, H, T and V in regulatory protein from Pseudomonas mendocina, and K, H, V and I in Vasotocin-neurophysin from Gallus gallus.

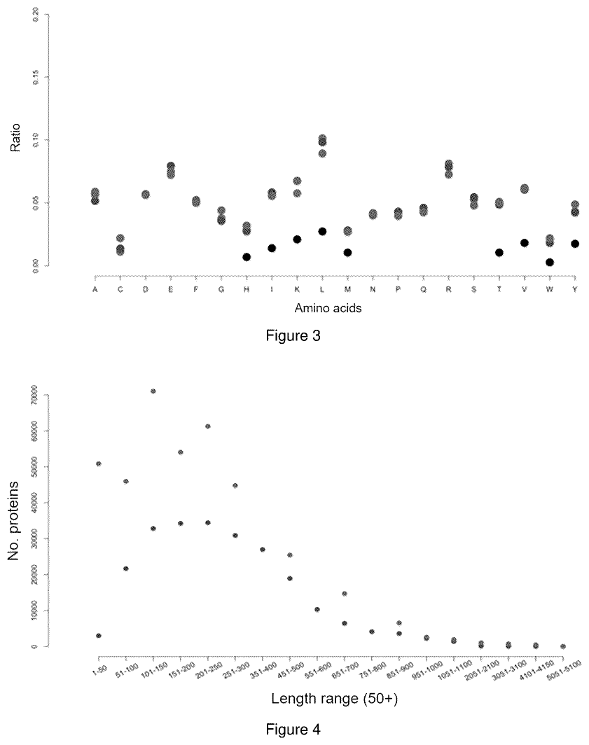

The ratios in which each of the 9 EAA was found in the 5 proteins found are shown in FIG. 2 of the accompanying drawings. As can be seen in FIG. 3, three (3) of the five (5) proteins found satisfied the requirement of AA, namely: threonine, phenylalanine, valine and isoleucine. The collagen protein from Bos taurus was the only one that satisfied the RDA for leucine, the uncharacterized protein from Macaca fascicularis the RDA for methionine. Vasotocin and the regulatory protein of Pseudomonas mendocina satisfied the RDA for lysine and histidine, but none of the five (5) proteins had the AA tryptophan in the ratio recommended by WHO. All of them lacked W, with the exception of the regulatory protein of Pseudomonas mendocina, which had 7-fold the RDA of W.

### Protein Mixtures Satisfying the Composition of 4 EAA in a 1× Ratio (Range 0.9-1.2).

When no protein was found that satisfied the RDA of the 9 EAA, the next step was to analyze whether there were any protein mixtures that had each of the 9 EAA in the required 1× ratio. The mixtures had up to 4 EAA in the recommended ratio (see Table 3).

TABLE 3
Protein mixtures identified in TrEMBL. A NAPR value of 4 and a range of 0.9-1.2
were used for Java code execution.

| | | | | Length | | |
|---|---|---|---|---|---|---|
| | | | | of AA of | | Total |
| | | | | the | No. of | protein |
| Protein 1 | Organism | Protein 2 | Organism | mixture | substitutions | change |
| Unknown | Homo sapiens | RNA binding | Acidovorax | 254 | 10 | 3.93% |
| protein | | protein | konjaci | | | |
| Hypothetical | Caenorhabditis | Hypothetical | Burkholderia | 246 | 22 | 8.94% |
| protein | latens | protein | pseudomallei | | | |
| FL83_14184 | | | | | | |
| Unchar- | Sporisorium | Hypothetical | Hypoxylon sp. | 237 | 27 | 11.39% |
| acterized | scitamineum | protein | CO27-5 | | | |
| protein | | M434DRAFT_ | | | | |
| SPSC_ | | 11978 | | | | |
| 00230 | | | | | | |
| Hypothetical | Rhizopus | Hypothetical | Octopus | 220 | 8 | 3.66% |
| protein | microsporus | protein | bimaculoides | | | |
| RMCBS344292_ | | OCBIM_ | | | | |
| 1658 | | 22014421mg | | | | |
| Hypothetical | Halobacteriales | Hypothetical | Cyphomyrmex | 244 | 13 | 5.32% |
| protein | archaeon | protein | costatus | | | |
| BRC95_ | | ALC62_09714 | | | | |
| 04495 | | | | | | |
| Hypothetical | Methanosarcina | Hypothetical | Halogranum | 186 | 15 | 8.06% |
| protein | mazei | protein | amylolyticum | | | |
| DU65_11645 | | | | | | |

As can be seen from Table 3 above, protein mixtures of 3 or more sequences did not satisfy the requirements of any of the 9 EAA and mixtures of 2 proteins had a very small percentage change from the original protein (about 5%). However, carrying out the necessary substitutions and expressing 2 proteins is more complicated than for a single protein, in addition to the fact that the mixtures generated correspond to proteins from the unreviewed Uniprot database that are uncharacterized and have not been previously expressed. Therefore, instead of choosing a mixture, we went back to evaluating whether one of the proteins previously found could be modified to have the EAA content in the ratio closest to that recommended for human consumption.

Of the 5 proteins that had been seen and that best approximated the EAA RDA, the one for which there was evidence that it was a protein, that its structure was known and that preferably had already been expressed was chosen. The protein that satisfied these characteristics was the collagen type XII protein from Bos taurus, which has a length of 86 AA and a molecular weight of 8 kDa. The Uniprot database shows that there is experimental evidence at the protein level and it is known to have stable structure.

Likewise, human type 1, 2, and 3 collagen fragments ranging from 8.7 to 43 kDa have previously been expressed in Pichia pastoris at high levels and secreted into the medium as single chains by the signal sequence of the yeast mating factor α (Nokelainen et al., 2001; Williams et al., 2008; He et al., 2015; Wang et al., 2014; Bin et al., 2011; Pakkanen et al., 2006) with yields up to 14.8 g/L (Werten et al., 1999).

The collagen protein would require 7 AA substitutions to have all the EAA in the proper ratio, so we proceeded to generate in silico sequence variants of this protein.

Generation of Collagen Protein Variants from Bos taurus.

The code developed in Java made it possible to find variants of the collagen protein by mutating the AA residues 20 by 20, with the exception of the amino acids proline and glycine. It was very interesting that no variant was found that had 1× the desired ratio, but only 20 variants were found that had 2 to 3 times the required ratio for the human daily intake, 9 of them with a percentage difference to the original collagen protein of 13.9% (see Table 4). 2 of these sequences could be chosen to evaluate the expression of the modified versions in Pichia pastoris.

TABLE 4
Bos taurus type XII collagen protein sequence variants. Of the 9
sequences found, the AA residues wherein the code introduced
substitutions regarding the WT sequence are shown in red and the
positions of the protein that the code could not mutate are marked
in blue.
WT sequence of collagen type XII protein from Bos taurus:
NQPGPPGPPGPPGSAGEPGPGGRPGFPGTPGMQGPQGERGLPGEXGERGLPGPPGPQ
GESRTGPPGSTGSRGPPGPPGRPGDSGIR
Collagen protein sequence variants of Bos taurus:
NKPGPPGPPGPPGSAFEPGPGGRPGFPGVPGMQGPQGERKLPGLMGERGLPGPPGPQG
ESRTIPPGSTGHRVPPGPPGRPGKLVIR
NQPGPPGPPGPPGSKGEPGPGGVPGFPGTPGMQMPQGERGLPGEXVKLHLPGPPGPQG
ESRFGPPGSTGSRIPPGPPGRPGDKVIL
NQPGPPGPPGPPGKAGVPGPGVRPGFPGKPGMQMIQGERGLPGLKGLVGLPGPPGPQG
ESRTGHPGSTGSRGPPGPPGRPGDFGIR
NKVGPPGPPGPPGSFGEPGPGGRPGFPGTPGMQGPQGVRGLPGMXGERLLPGPPGPQ
GESRTIKPGSKHSRGPPGPPGRPGVSLIR
MFPGPPGPPGPPGSKGHPGPGLRPGFPGTPGMQGVQKEVGLPGEXGVRGLPGPPGPQG
ESRTGPPGSKGSRGIPGPPGRPGDLGIR
HQPGPPGPPGPPGLAGKPGPVGKPGFPGTPGMQGPQVERMLPGEVGLRGLPGPPGPQG
ESKFGPPGSTISRGPPGPPGRPGDSGIR
NQPGPPGPPGPPGSLVLPGPGGRPGFPGTPGMQGPQKEVGLPGEKGEKVLPGPPGPQG
ESRIGMPGSTGSHGFPGPPGRPGDSGIR
NQPGPPGPPGPPGKAGEPGPGGRPGFPGTPGMQGPKFERGLPGEXKERVLPGPPGPQG
ESVTGIPGSLGSRLPPGPPGRPGHVGIM
NQPLPPGPPGPPGSKVEPGPGGRPGFPGVPGMQGPKGERGLPGHXVELGLPGPPGPQG
ESRTGFPGSTKSRIPPGPPGRPGDMGIR

No tryptophan (W) was introduced to any variant of the collagen protein because the required ratio of W was the lowest (0.0028) and the length of the collagen protein was only 86 AA. This meant that the ratio of W to be satisfied was less than 1 tryptophan in the protein, so it was not possible to find variants with 1× the required ratio of this EAA.

### Proteins Having 2 to 3 Times the Desired EAA Composition (Range 1.9-3.1) in Uniprot and PDB.

When generating the collagen variants it was observed that it was not possible to generate a sequence that had exactly 1× the required composition of each EAA, so we went back to search in the databases if there were proteins that had 2 to 3 times the required ratio of each EAA and not just once (1×) as is the case for the Bos taurus collagen protein (range 0.9-1.2). It was found that there are no sequences that satisfy all 9 EAA ratios in the range of 1.9 to 3.1; however, there are 2 proteins in the SwissProt database that satisfy the requirement for up to 8 EAA (with the exception of tryptophan) in a ratio of 2-3×:
RS15_NATPD 30S ribosomal protein RS15_NATPD from Natronomonas pharaonis (Uniprot ID: Q3IQA3). The W was at a ratio of 3.7×:
Pre-mRNA splicing factor CWC21 of Ashbya gossypii (Uniprot ID: Q751G9). W was at a ratio of 0×.

In TrEMBL, 141 sequences were found that had 8 EAA ranging from 1.9-3.1. All of these proteins had an EAA outside the desired ratio, some of them are as shown in Table 5 below:

TABLE 5
Proteins noted in TrEMBL that satisfy 2 to 3 times the
required human intake of EAA. A NAPR value of 8, a range
of 1.9-3.1 and a maximum length of 260 AA were used.

| | | | Protein | EAA out |
|---|---|---|---|---|
| | | | length | of ratio |
| ID Uniprot | Protein | Organism | (AA) | (1.9-3.1) |
| A0A091CL33 | Uncharac- | Fukomys | 230 | W = 7.6x |
| | terized | damarensis | | |
| | protein | | | |
| A0A1U8DU65 | Ribonu- | Alligator | 245 | M = 3.6x |
| | cleoprotein- | sinensis | | |
| | associated | | | |
| | protein | | | |
| A0A1A8NEZ1 | SH3 domain | Nothobranchius | 207 | F = 3.7x |
| | binding | pienaari | | |
| | glutamic | | | |
| | acid-rich | | | |
| | protein | | | |
| A0A0S7ENX5 | RNF37 | Poeciliopsis | 210 | W = 11.4x |
| | (Fragment) | prolifica | | |
| M4AJT2 | Intraflagellar | Xiphophorus | 210 | W = 8.2x |
| | transport 43 | maculatus | | |
| K7G7M7 | Syntaxin 7 | Pelodiscus | 258 | I = 7x |
| | | sinensis | | |
| A0A2K5NP26 | Uncharac- | Cercocebus | 223 | F = 10.3x |
| | terized | atys | | |
| | protein | | | |
| A0A2K5ZMJ3 | Uncharac- | Mandrillus | 223 | F = 10.4x |
| | terized | leucophaeus | | |
| | protein | | | |
| J3MH86 | Uncharac- | Oryza | 212 | H = 0x |
| | terized | brachyantha | | |
| | protein | | | |
| A0A2H5N6J0 | Uncharac- | Citrus unshiu | 72 | W = 16.4x |
| | terized | | | |
| | protein | | | |
| A0A287MZH9 | Uncharac- | Hordeum | 240 | T = 6x |
| | terized | | | |
| | protein | vulgare | | |

### Proteins Having 3 to 4 Times the Desired EAA Composition (Range 2.9-4.1).

In SwissProt, 8 proteins were found to be missing only one EAA in the 3-4× ratio, as can be seen in Table 6 below:

TABLE 6
SwissProt annotated proteins satisfying a ratio of 3-4x. A NAPR value
of 8 and a range of 2.9-4.1 was used for Java code execution.

| | | | Protein | EAA out |
|---|---|---|---|---|
| | | | length | of ratio |
| ID Uniprot | Protein | Organism | (AA) | (2.9-4.1) |
| Q9VR89 | RNA-binding | Drosophila | 240 | W = 2.4x |
| | protein pno 1 | melanogaster | | |
| A5VIU4 | 30S ribosomal | Lactobacillus | 201 | F = 5.4x |
| | protein S4 | reuteri | | |
| Q0C187 | Methyl- | Hyphomonas | 234 | W = 7.7x |
| | transferase E | neptunium | | |
| Q7VS88 | Deformylase 2 | Bordetella | 170 | V = 5x |
| | | pertussis | | |
| Q87TT1 | ATP synthase | Pseudomonas | 178 | L = 4.2x |
| | delta subunit | syringae | | |
| | | pv. tomato | | |
| Q9D898 | Actin-related | Mus musculus | 153 | M = 2.7x |
| | protein | | | |
| Q089E2 | LexA repressor | Shewanella | 204 | I = 5.3x |
| | | frigidimarina | | |
| P30334 | Ribosomal | Bradyrhizobium | 203 | F = 4.5x |
| | hibernation factor | diazoefficiens | | |

Proteins that Complement Natural Protein Sources: Eggs, Milk, Beef, Pork, Fish and Chicken.

We searched for proteins that could serve as a dietary supplement for athletes, older adults and people with innate protein metabolism diseases. For this purpose, proteins were sought to complement the EAA content of the natural sources in a 1× ratio.

However, none of the proteins found were good candidates because they had only 3 of the 9 EAA in the recommended ratio, as shown in Table 7 below:

TABLE 7
Proteins annotated in PDB that complement egg,
milk and fish in a 1x ratio. NAPR values (2-3)
and a range of 0.9-1.2 for Java code execution.

| | | | | EAA within |
|---|---|---|---|---|
| | Length | | Complemented | the required |
| Protein | (AA) | ID PDB | protein source | ratio 1x |
| Vasopressin V1a | 84 | 1ytv_M | Egg | 2 |
| receptor | | | | |
| Ribosomal | 85 | 5it7_jj | Egg | 2 |
| protein L37 | | | | |
| Art v1 pollen | 108 | 2kpy_A | Fish | 2 |
| allergen | | | | |
| Phospholipase A2 | 119 | 1mh2_B | Milk | 3 |
| Filamin-A | 95 | 2mtp_A | Milk | 3 |
| Interleukin 11 | 177 | 4mhl_A | Milk | 3 |
| Anticoagulant | 85 | 1cou_A | Milk | 2 |
| protein C2 | | | | |
| Xylanase D | 87 | 1e5b_A | Milk | 2 |

The proteins found in PDB (Table 7) supplemented egg and fish with up to 2 EAA in the 1× ratio required in human daily intake; whereas proteins supplementing milk do so with up to 3 EAA in this required ratio.

On the other hand, in PDB, proteins were found to satisfy the requirements of a greater number of EAA as the required ratio increased (2-4×), as shown in Tables 8 and 9 below:

TABLE 8
Proteins noted in PDB that complement egg, milk, chicken,
fish, beef and pork in a 2-3x ratio. NAPR values (2-6)
and a range of 1.9-3.1 for Java code execution.

| | | | | EAA within |
|---|---|---|---|---|
| | Length | | Complemented | the required |
| Protein | (AA) | ID PDB | protein source | ratio 2-2x |
| Ribosomal protein | 94 | 4v8p_AA | Beef | 3 |
| Helical repeat | 239 | 5cwm_A | Beef | 3 |
| protein | | | | |
| Ethanol regulon | 65 | 1f4s_P | Chicken | 2 |
| transcriptional | | | | |
| factor | | | | |
| Vasopressin V1a | 84 | 1ytv_M | Chicken | 2 |
| receptor | | | | |
| Muscarinic toxin- | 65 | 3hh7_A | Chicken | 2 |
| like protein | | | | |
| homologue | | | | |
| Neurotrophin-4 | 130 | 1b8m_B | Egg | 4 |
| Pleiotropin | 136 | 2n6f_A | Egg | 4 |
| Endoxylanase | 151 | 1o8p_A | Egg | 4 |
| L37 60S ribosomal | 97 | 4ug0_Lj | Fish | 3 |
| protein | | | | |
| Prokaryotic | 68 | 3m9d_G | Milk | 6 |
| ubiquitin-like | | | | |
| protein | | | | |
| ESAT-6-like protein | 94 | 4i0x_A | Milk | 6 |
| MAB_3112 | | | | |
| Rab-3A-interacting | 78 | 4lhx_C | Milk | 6 |
| protein | | | | |
| Streptavidin | 159 | 5f2b_A | Milk | 6 |
| Uncharacterized | 139 | 4lmi_A | Pork | 3 |
| protein | | | | |

TABLE 9
Proteins noted in PDB that complement beef, chicken, egg, fish, milk and pork in
a 3-4 x ratio. NAPR values (2-3) and a range of 2.9-4.1 for Java code execution.

| | | | | EAA within |
|---|---|---|---|---|
| | | | Complemented | the required |
| Protein | Length (AA) | ID PDB | protein source | ratio 3-4x |
| Phospholipase A2 | 122 | 1faz_A | Beef | 3 |
| Matrix | 72 | 1j7m_A | Beef | 3 |
| metalloproteinase 2 | | | | |
| Small nuclear | 61 | 4pjo_L | Beef | 3 |
| ribonucleoprotein C | | | | |
| Coagulation factor IX- | 123 | 1j34_B | Chicken | 2 |
| binding protein B chain | | | | |
| Ribosomal protein | 66 | 1m1k_V | Chicken | 2 |
| L24E | | | | |
| Tachylectin 2 | 136 | 1tl2_A | Egg | 5 |
| Restriction | 166 | 3zi5_A | Egg | 5 |
| endonuclease K | | | | |
| Eukaryotic translation | 128 | 5h7u_A | Egg | 5 |
| initiation factor 3 | | | | |
| Endoglucanase | 181 | 1wc2_A | Fish | 3 |
| Nuclear small | 61 | 4pjo_I | Fish | 3 |
| ribonucleoprotein C | | | | |
| 5(3) | 197 | 1q91_A | Milk | 7 |
| deoxyribonucleotidase | | | | |
| prgH protein | 227 | 3gr1_G | Milk | 7 |
| Na(⁺)/H(⁺) exchange | 210 | 2krg_A | Milk | 7 |
| regulatory cofactor | | | | |
| NHE-RF1 | | | | |
| Hsp 90-associated | 110 | 2lsu_A | Milk | 7 |
| protein | | | | |
| Agglutinin isolectin I | 89 | 1en2_A | Pork | 3 |
| Collagen alpha-1 (XX) | 104 | 2dkm_A | Pork | 3 |
| chain | | | | |
| eL29 | 245 | 5lzw_b | Pork | 3 |

After analyzing the proteins that complemented the natural sources in 1×, 2-3× and 3-4× ratios, it was found that the best complementing feed was milk with up to 7 EAA in the 3-4× ratio. The proteins that supplemented it had the EAA phenylalanine and tryptophan out of this ratio with up to 8 to 9 times the required ratio of phenylalanine. The NHE-RF1 cofactor had the EAA lysine (4.4×) and methionine (4.3×) out of the 3-4× ratio. In the TrEMBL database, no proteins complementing beef, pork, fish and chicken with NAPR values of 2-9 were found.

In SwissProt it was observed that the protein supplementing beef satisfied the requirement of 3 EAA in a 1× ratio. For milk, no protein supplemented its EAA content, for pork and chicken only 1 EAA was satisfied, while for egg and fish 2 EAA were satisfied, as can be seen in Table 10 below:

TABLE 10
SwissProt proteins that complement beef, chicken, egg and fish in a 1x ratio.
NAPR values (1-3) and a range of 0.9-1.2 for Java code execution.

| | | | | | EAA within |
|---|---|---|---|---|---|
| | | | | Protein | the required |
| Protein | Organism | Length (AA) | ID | source | ratio 1x |
| Late | Arabidopsis | 225 | Q9LW12 | Beef | 3 |
| embryogenesis | thaliana | | | | |
| abundant protein | | | | | |
| 29 | | | | | |
| Spore coat | Bacillus subtilis | 195 | P39801 | Beef | 2 |
| protein G | | | | | |
| Tumor | Homo sapiens | 212 | Q2TAM9 | Beef | 2 |
| suppressor | | | | | |
| protein | | | | | |
| Neuromodulin | Bos taurus | 242 | P06836 | Chicken | 1 |
| Keratin- | Mus musculus | 130 | Q9Z287 | Chicken | 1 |
| associated | | | | | |
| protein 12-1 | | | | | |
| Phospholemman | Oryctolagus | 92 | G1TZA0 | Egg | 2 |
| | cuniculus | | | | |
| Nuclear | Sus scrofa | 137 | P29258 | Egg | 2 |
| transition protein | | | | | |
| 2 | | | | | |
| Myelin- | Bos taurus | 195 | 1e5b_A | Egg | 2 |
| associated | | | | | |
| neurite | | | | | |
| outgrowth | | | | | |
| inhibitor | | | | | |
| Uncharacterized | Acanthamoeba | 195 | Q5UQT4 | Fish | 2 |
| protein R346 | polyphaga | | | | |
| | mimivirus | | | | |

Not finding proteins with more than 3 EAA in a 1× ratio, we searched for proteins in SwissProt that would complement the natural sources with a ratio of 2 to 3 times higher than recommended.

TABLE 11
SwissProt annotated proteins that complement natural protein sources in a 2-3x
ratio. NAPR values (3-5) and a range of 1.9-3.1 for Java code execution.

| | | | | | EAA within |
|---|---|---|---|---|---|
| | | | | Protein | the required |
| Protein | Organism | Length (AA) | ID | source | ratio 2-3x |
| Late | Arabidopsis | 169 | Q96270 | Beef | 3 |
| embryogenesis | thaliana | | | | |
| abundant | | | | | |
| protein 7 | | | | | |
| M-phase- | Mus musculus | 178 | Q9D011 | Beef | 3 |
| specific PLK1- | | | | | |
| interacting | | | | | |
| protein | | | | | |
| MARCKS- | Oryctolagus | 199 | G1TZA0 | Beef | 3 |
| related protein | cuniculus | | | | |
| Nuclear | Sus scrofa | 137 | P29258 | Chicken | 3 |
| transition | | | | | |
| protein 2 | | | | | |
| Glycine-rich | Oryza sativa | 166 | A3CG83 | Egg | 5 |
| cell wall | subspecies | | | | |
| structural | japonica | | | | |
| protein | | | | | |
| Submandibular | Rattus | 246 | P08568 | Egg | 5 |
| gland secretory | norvegicus | | | | |
| Glycine-rich | | | | | |
| protein CA | | | | | |
| L37 60 S | Bos taurus | 97 | P79244 | Fish | 3 |
| ribosomal | | | | | |
| protein | | | | | |
| L37 60 S | Homo sapiens | 97 | P61928 | Fish | 3 |
| ribosomal | | | | | |
| protein | | | | | |
| M-phase- | Mus musculus | 178 | Q9D011 | Fish | 3 |
| specific PLK1- | | | | | |
| interacting | | | | | |
| protein | | | | | |
| Secretin | Homo sapiens | 121 | P09683 | Pork | 4 |

As could be seen from Table 11 above, in SwissProt it was observed that there were proteins that supplemented the EAA content of the egg with up to 5 EAA in a ratio of 2-3×. One of them, the glycine-rich structural protein from the cell wall of Oryza sativa could be a candidate protein as a dietary supplement. For the case of pork complementing proteins, secretin from Homo sapiens was found to have up to 4 EAA in the ratio of 2-3×. For beef, chicken and fish, results were found with a NAPR value of 3, while for milk there were no complementing proteins. Some of the proteins that supplemented the food at a ratio of 2-3× had also been found to supplement it at a ratio of 1×. An example is nuclear transition protein 2 from Sus scrofa, which had been found to complement egg with a NAPR value of 2 at a 1× ratio, in addition to complementing chicken with a NAPR value of 3 at a ratio of 2-3×. The proteins found in both 1× and 2-3× ratios correspond to the same organisms: Bos taurus, Rattus norvegicus, Sus scrofa, Homo sapiens, Mus musculus, Oryctolagus cuniculus and Arabidopsis thaliana. In addition to the above, 2 abundant late embryogenesis proteins from Arabidopsis thaliana, 29 and 7, complemented the same food, beef in 1× and 2-3× ratios, respectively.

Proteins from Bos taurus, Homo sapiens and Streptococcus pneumoniae complemented the egg with up to 5 EAA in a ratio of 3-4× (see Table 12). The EAA content of the pork was supplemented by a protein with up to 4 EAA in the recommended ratio. For fish, proteins from various organisms were found, ranging from archaea, viruses and bacteria to proteins from Bos taurus, Gallus gallus, Danio rerio, Oryctolagus cuniculus, Rattus norvegicus and Xenopus tropicalis with a NAPR value of 3.

TABLE 12
SwissProt annotated proteins that complement natural protein sources in a 3-4x
ratio. NAPR values (3-5) and a range of 2.9-4.1 for Java code execution.

| | | | | | EAA within the |
|---|---|---|---|---|---|
| | | | | Protein | required ratio |
| Protein | Organism | Length (AA) | ID | source | 2-3x |
| L35 50S ribosomal protein | Lactobacillus | 169 | Q5FLW8 | Beef | 3 |
| | acidophilus | | | | |
| Hsp9 Heat Shock Protein | S. pombe | 68 | P50519 | Beef | 3 |
| Keratin-associated protein | Mus musculus | 150 | Q9QZU5 | Beef | 3 |
| 15-1 | | | | | |
| Chromosomal protein 6 | Emericella | 106 | Q5BP05 | Chicken | 3 |
| | nidulans | | | | |
| Uncharacterized protein | S. cerevisiae | 128 | P38216 | Chicken | 3 |
| YBR16W | | | | | |
| Serine/arginine rich splicing | Homo sapiens | 238 | Q16629 | Egg | 5 |
| factor 7 | | | | | |
| Subunit delta RNA S | Streptococcus | 200 | P66718 | Egg | 5 |
| polymerase | pneumoniae | | | | |
| Nuclear protein 2 | Bos taurus | 98 | Q32PB4 | Egg | 5 |
| Tegument protein | Epstein Barr virus | 217 | P0C724 | Fish | 3 |
| E3 ubiquitin-protein ligase | Xenopus tropicalis | 119 | Q6GLB0 | Fish | 3 |
| PPP1R11 | | | | | |
| Protein phosphatase 1 | Oryctolagus | 166 | P01099 | Fish | 3 |
| regulatory subunit 1A | cuniculus | | | | |
| Proline-rich cell wall protein | Glycine max | 230 | P13993 | Fish | 3 |
| 2 | | | | | |
| Transglycosylase SceD 1 | Staphylococcus | 238 | Q4A0X5 | Fish | 3 |
| | saprophyticus | | | | |
| Small cellular | Danio rerio | 159 | Q8JGS0 | Fish | 3 |
| ribonucleoprotein C | | | | | |
| High mobility group protein | Gallus gallus | 202 | P40618 | Fish | 3 |
| B3 | | | | | |
| INO80 complex subunit E | Bos taurus | 244 | Q29RS4 | Fish | 3 |
| Submandibular gland | Rattus norvegicus | 246 | P08568 | Fish | 3 |
| secretory Glycine-rich | | | | | |
| protein CA | | | | | |
| ATP synthase subunit B | Staphylothermus | 195 | A3DNQ9 | Fish | 3 |
| | marinus | | | | |
| G-protein-signaling | Rattus norvegicus | 158 | Q6MG88 | Pork | 4 |
| modulator 3 | | | | | |

Ratios in which Each AA is Found in the Proteins of the PDB and Uniprot Databases (SwissProt and TrEMBL).

The ratios in which each AA was found in the proteins annotated in PDB, SwissProt and TrEMBL were searched to see if this ratio was higher or lower than that required in the human daily intake, and thus, in this way, to analyze why the proteins found did not satisfy the requirements of the 9 EAA.

The ratios of each of the 20 AA were very similar in the 3 databases analyzed (see FIG. 3 of the accompanying drawings). The AA found in lower ratios were tryptophan, cysteine, methionine and histidine. Leucine, glutamic acid and arginine were in higher ratios. To obtain the graph shown in FIG. 3, the 20 AA (A, C, D, E, F, G, H, I, K, L, M, N, P, Q, R, S, T, V, W, Y) were taken into account. The WHO recommended ratios of each of the 9 EAA, PDB (red dots), TrEMBL (purple dots) and SwissProt (blue dots) are shown in black dots.

The ratios in which each AA was found in SwissProt, PDB and TrEMBL, as well as the WHO recommended ratios for each EAA, are shown in Table 13. The ratio of each AA was equal to the number of times the AA was found in the annotated proteins*average mass of the AA/average mass of the annotated proteins. The ratios are normalized to 1 g of protein. In red numbers are the AA that were found in lower ratio and in blue the other AA are shown: F, T, I, V, K and L.

TABLE 13
Ratios of each AA in 1 gram of protein.

| | | | | | Average of the ratio of |
|---|---|---|---|---|---|
| | | | | | each AA in the 3 |
| AA | OMS | SwissProt | PDB | TrEMBL | databases |
| C | | 0.0136 | 0.0219 | 0.0111 | 0.0155 |
| W | 0.0028 | 0.0181 | 0.0215 | 0.0219 | 0.0205 |
| M | 0.0105 | 0.0278 | 0.0268 | 0.0281 | 0.0275 |
| H | 0.0070 | 0.0282 | 0.0319 | 0.0272 | 0.0291 |
| G | | 0.0357 | 0.0440 | 0.0377 | 0.0391 |
| N | | 0.0415 | 0.0418 | 0.0399 | 0.0410 |
| P | | 0.0430 | 0.0396 | 0.0427 | 0.0417 |
| Q | | 0.0460 | 0.0424 | 0.0438 | 0.0440 |
| Y | | 0.0422 | 0.0487 | 0.0430 | 0.0446 |
| T | 0.0105 | 0.0485 | 0.0500 | 0.0508 | 0.0497 |
| F | 0.0175 | 0.0506 | 0.0499 | 0.0523 | 0.0509 |
| S | | 0.0545 | 0.0479 | 0.0525 | 0.0516 |
| A | | 0.0515 | 0.0564 | 0.0588 | 0.0555 |
| D | | 0.0563 | 0.0562 | 0.0570 | 0.0565 |
| I | 0.0140 | 0.0580 | 0.554 | 0.0583 | 0.0572 |
| V | 0.0182 | 0.0604 | 0.0611 | 0.0619 | 0.0611 |
| K | 0.0210 | 0.0675 | 0.0673 | 0.0575 | 0.0641 |
| E | | 0.0793 | 0.747 | 0.0721 | 0.0753 |
| R | | 0.083 | 0.0724 | 0.0812 | 0.0773 |
| L | 0.0273 | 0.980 | 0.0891 | 0.1013 | 0.0961 |

The mean of the ratios of the AA was 0.047, which showed that the AA appeared uniformly. It was observed that, although the ratios of tryptophan (0.0205), methionine (0.0275) and histidine (0.0291) were among the lowest in the databases, they were still higher than those required for human daily intake (0.0028 for tryptophan, 0.0105 for methionine and 0.0070 for histidine). Thus, the requirement for a greater number of AA at higher ratios (2-4×) was satisfied. In addition to obtaining the AA ratios, the code also yielded the number of proteins falling within a length range, which was increasing by 50 AA, as shown in FIG. 4 of the accompanying drawings.

As can be seen in FIG. 4, sequences of 1-50 AA (50,830), 51-100 AA (45,928), 101-150 AA (71,031), 151-200 AA (54,033) and 201-250 AA (61,246) are mostly annotated in PDB. The number of proteins found decreased in longer AA length ranges, with proteins from 4851-4900, 4301-4350, 3951-4000, 3801-3850 and 2951-3000 AA being more difficult to find, all with only one protein found within those ranges.

In PDB there were 50,830 peptides from 1-50 AA, whereas in SwissProt there were only 2,988 in this range. As in the PDB database, a clear downward trend was observed in SwissProt in the number of proteins with longer AA lengths, as is the case for the ranges 4901-4950 (5), 4601-4650 (6) and 4851-4900 (6).

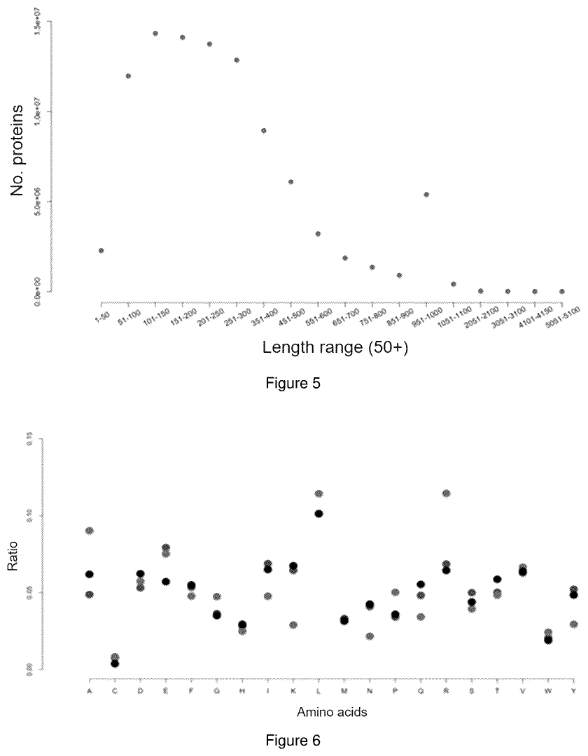

In the TrEMBL database (see FIG. 5 of the accompanying drawings), it can be seen that there are more proteins from 101-150 AA (14,327,667), followed by proteins in the range 151-200 (14,108,345) and 201-250 (13,734,838). There are a greater number of proteins in the 951-1000 AA range (5,384,100) and in smaller ratio are proteins within the 5051-5100 (911), 5001-5050 (1262), 4851-4900 (1281) and 4901-4950 (1322) ranges.

Of the 3 length distributions analyzed (PDB, SwissProt and TrEMBL), it can be observed that 1-250 AA sequences are abundant. Restricting the search to proteins up to 260 AA caused the required ratios of EAA such as W, M and H to be even lower. An example of this is the case of tryptophan, which is required in a ratio of 0.0028. In proteins up to 260 AA this ratio is not satisfied because the requirement does not even reach 1 tryptophan per protein, while the ratio of W in proteins is higher than required.

Tryptophan is the EAA that is required in the lowest ratio (0.0028) but also has the largest mass (186.2132 Da). The following probabilistic calculation was used to determine the possible minimum length that the protein should have to satisfy the requirement of each EAA, taking into account the average mass of the 20 EAA:

$$P / n * 118.736 = V$$

wherein:
P is the average atomic mass of the EAA;
n is the minimum protein length to satisfy the required EAA ratio;
V is the required ratio of EAA as reported by WHO; and
118.7360 is the average mass of the 20 AA.

By applying the above formula for the case of tryptophan (n=186.2132/(118.7360*0.0028)), the possible minimum length to satisfy its required ratio (0.0028) was found to be 560 AA. Therefore, it was unlikely that the 9 EAA in the recommended ratios using a sequence search range of 1-260 AA. For the other EAA, the minimum protein lengths to satisfy their requirement are shown in Table 14 below:

TABLE 14
Minimum protein length to satisfy the requirements of the
9 EAA (W, K, H, T, M, V, I, L, F). The possible minimum length
of proteins to satisfy the requirement of each EAA was obtained
by probabilistic calculation: P/n*118.7360 = V.

| | Minimum length of |
|---|---|
| | protein to meet the |
| EAA | requirement |
| W | 560 |
| K | 51 |
| H | 165 |
| T | 81 |
| M | 105 |
| V | 46 |
| I | 68 |
| L | 40 |
| F | 71 |

As can be seen in Table 14, using the length range of 1-260 AA, the requirement for 8 EAA was satisfied, with the exception of tryptophan. In proteins up to 1000 AA, the requirement for more than 4 EAA in the 1× ratio was also not satisfied. One possible explanation for this is that the probability of finding proteins with all the AA in the ratio recommended by the WHO is very low. Length distributions showed that there were about 3×10\hat{ }5 sequences in SwissProt and 4.5×10\hat{ }5 sequences in PDB of 1-1000 AA. For a 560 AA sequence to have a tryptophan, one would have to find all possible 560 AA sequences that did not have W (19\hat{ }560 sequences) and add a W to each. The probability of finding such sequences was 1/19\hat{ }559 and in a sample of 3-4.5×10\hat{ }5 sequences this was very unlikely.

### Ratios of Each AA at the Proteome Level.

After having seen the ratios in which AA were found in the databases, these ratios were analyzed at the proteome level. Proteomes of fungi, plants, animals, bacteria and viruses were chosen to see if the human diet could be inducing a trend in the EAA composition of the organisms, with ratios closer to those required for the case of proteomes of organisms from which our food production is based such as beef, pork and chicken. The proteomes analyzed were as follows:
Bacteria: Paenibacillus polimyxa (Gram-positive bacteria capable of fixing nitrogen, used in biocontrol and antibiotic production), Rhodobacter spheroides (photosynthetic purple bacteria of diverse metabolism) and Lactobacillus casei (probiotic anaerobic bacteria).
Fungi: Tuber melanosporum (known as truffle, it is highly appreciated in gastronomy and of great economic value), Ustilago maydis (known colloquially as huitlacoche, it is a traditional part of Mexican food), Schizophyllum commune (saprophytic fungus of deciduous tree trunks) and Saccharomyces cerevisiae (yeast for industrial use in the manufacture of bread, beer and wine).
Plants: Glycine max (known as soybean, it is widely used in human food), Oryza sativa (provides 20% of the world's dietary energy supply (FAO)).
Animals: Acynonyx jubatus (cheetah), Alligator mississippiensis (Mississippi alligator), Bos taurus (cow), Camelus dromedarius (Arabian camel), Gadus morhua (Norwegian cod), Gallus gallus (chicken) and Sus scrofa (pig).
Viruses: influenza A virus, human immunodeficiency virus 1 and SARS-COV-2.

FIG. 6 of the accompanying drawings shows the AA ratios of bacterial proteomes, wherein the ratio of each AA is equal to the number of times the AA was found in the annotated proteins in the database*average mass of the AA/average mass of the annotated proteins. The 20 AA were taken into account. The ratio in the proteomes of Paenibacillus polymyxa (blue dots), Rhodobacter spheroides (purple dots) and Lactobacillus casei (black dots).

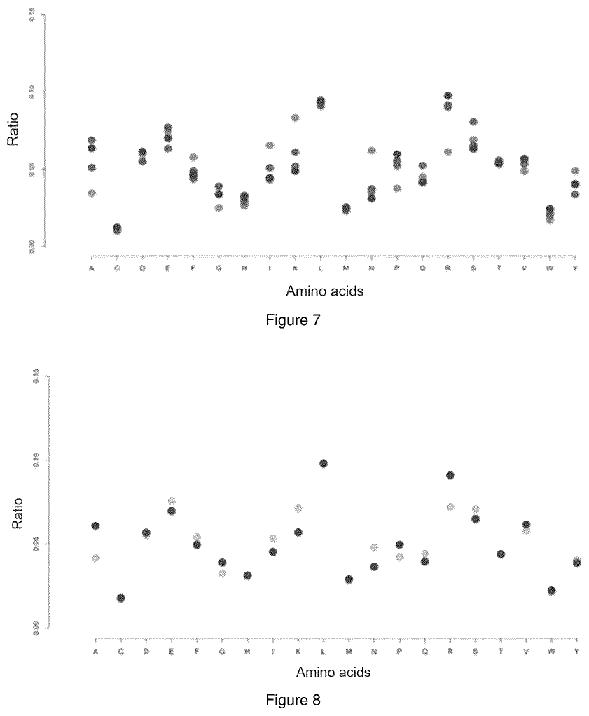

FIG. 7 of the accompanying drawings shows the AA ratios of fungal proteomes, wherein the ratio of each AA is equal to the number of times the AA was found in the annotated proteins in the database*average mass of the AA/average mass of the annotated proteins. The 20 AA were taken into account. The ratio in the proteomes of Tuber melanosporum (red dots), Ustilago maydis (purple dots), Schizophyllum commune (blue dots) and Saccharomyces cerevisiae (pink dots).

FIG. 8 of the accompanying drawings shows the AA ratios of plant proteomes, wherein the ratio of each AA is equal to the number of times the AA was found in the annotated proteins in the database*average mass of the AA/average mass of the annotated proteins. The 20 AA were taken into account. The ratio in the proteomes of Glycine max (green dots) and Oryza sativa (brown dots).

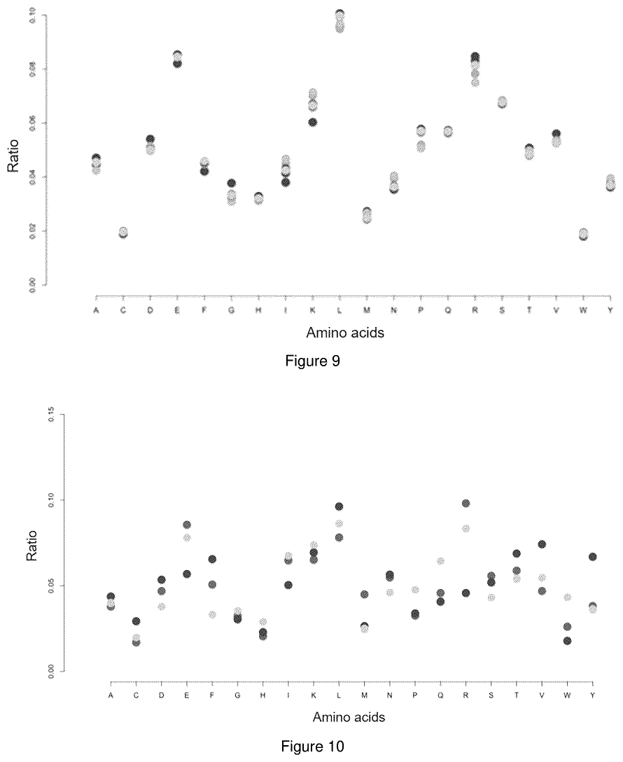

FIG. 9 of the accompanying drawings shows the AA ratios of animal proteomes, wherein the ratio of each AA is equal to the number of times the AA was found in the annotated proteins in the database*average mass of the AA/average mass of the annotated proteins. The 20 AA were taken into account. The ratio in the proteomes of Acynonyx jubatus (dots in black color), Alligator mississippiensis (dots in green color), Bos taurus (dots in red color), Camelus dromedarius (dots in brown color), Gadus morhua (dots in blue color), Gallus gallus (dots in orange color) and Sus scrofa (dots in pink color).

FIG. 10 in the accompanying drawings shows the AA ratios of virus proteomes, wherein the ratio of each AA is equal to the number of times the AA was found in the annotated proteins in the database*average mass of the AA/average mass of the annotated proteins. The 20 AA were taken into account. The ratio in the proteomes of influenza A virus (purple dots), human immunodeficiency virus 1 (pink dots) and SARS-COV-2 (blue dots).

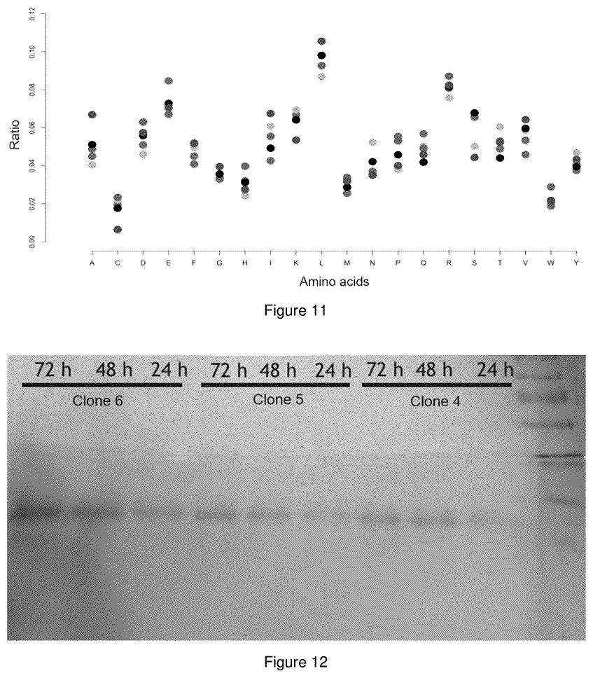

FIG. 11 in the accompanying drawings shows the ratios of AA in the proteomes of animals, plants, fungi, viruses and bacteria, wherein the ratio of each AA is equal to the number of times the AA was found in the annotated proteins in the database*average mass of the AA/average mass of the annotated proteins. The 20 AA were taken into account. The ratio in proteomes of animals (purple dots), plants (black dots), fungi (red dots), viruses (gray dots) and bacteria (blue dots).

TABLE 15
Average ratio and standard deviation of each AA in the 19 proteomes
analyzed. The average ratio and standard deviation were calculated
for each of the 20 AA from the ratios of the proteomes of animals
(7), plants (2), fungi (4), viruses (3) and bacteria (3).

| | | Standard |
|---|---|---|
| AA | Average ratio | deviation |
| A | 0.0491 | 0.0152 |
| C | 0.0184 | 0.0112 |
| D | 0.542 | 0.0072 |
| E | 0.0756 | 0.0100 |
| F | 0.0467 | 0.0080 |
| G | 0.0344 | 0.0042 |
| H | 0.0315 | 0.0087 |
| I | 0.0517 | 0.0119 |
| K | 0.043 | 0.0125 |
| L | 0.0927 | 0.0175 |
| M | 0.0296 | 0.0090 |
| N | 0.0393 | 0.0077 |
| P | 0.0487 | 0.0088 |
| Q | 0.0508 | 0.0083 |
| R | 0.0816 | 0.0143 |
| S | 0.0607 | 0.0107 |
| T | 0.0516 | 0.0057 |
| V | 0.0549 | 0.0108 |
| W | 0.0232 | 0.0082 |
| Y | 0.0403 | 0.0080 |

By analyzing the proteomes, it can be observed that AA compositions are conserved across all organisms. The standard deviation values for each of the 20 AA showed that the dispersion of the values regarding the average ratio was very low. The ratios in which AA were found were very similar among the different kingdoms (plants, animals, prokaryotes and fungi), as well as in viruses. On average, the ratio of each AA deviated from the mean between 0.0057 and 0.0175. The AA tryptophan, methionine, histidine and cysteine were in lower ratio and leucine, arginine and glutamic acid in higher ratio.

Strains of the yeast Pichia pastoris X33 overexpressing the plasmid carrying the gene coding for the protein of SEQ ID NO. 16 were isolated using different concentrations of the antibiotic Zeocin (400 μg/mL, 800 μg/mL and 1200 μg/mL). Two strains were isolated that grew at the highest concentration of Zeocin expressing the protein gene of SEQ ID NO. 16 under the pAOX1 promoter and 3 strains that grew at the highest concentration of Zeocin expressing the protein of SEQ ID NO. 16 under the promoter pGAP.

These 5 selected strains were grown in rich medium (YPD: yeast extract, peptone and Glucose) for 18 hours, and then transferred to a 250 mL volume flask containing 50 mL of PTM1 salts medium at pH 4.0 with 2% glucose for strains with the pGAP promoter and 1.5% methanol for strains with the pAOX1 promoter. They were grown for 96 hours at 25° C. with 250 rpm agitation; samples were taken every 24 hours from these cultures. To verify that these strains produced the protein, samples were centrifuged at 5,000 rpm and the cells in the pellet generated were discarded. The supernatant from these centrifugations was used to load denaturing polyacrylamide gels and stained with Coomasie blue.

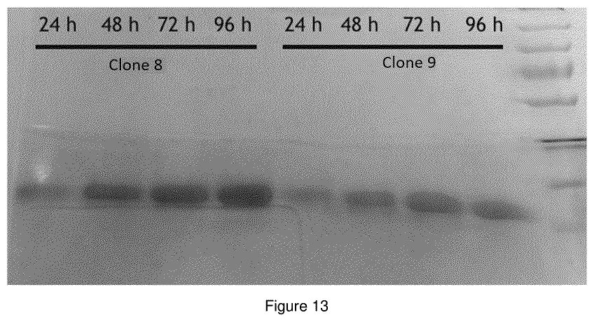

FIG. 12 of the accompanying drawings shows the results of those gels for the 3 strains with the pGAP promoter, while FIG. 13 of the accompanying drawings shows the 2 strains with the pAOX1 promoter. In both FIGS. 12 and 13, bands of different colors corresponding to molecular weight markers (PageRuler™ from ThermoFisher Catalog No. 26619) can be seen on the far right. The expected molecular weight of the protein is 8 KDa. The two lower bands in the molecular weight marker correspond to 10 and 15 KDa proteins. It is noted that the native Pichia pastoris X33 strain that was not transfected with the plasmid encoding for the protein of SEQ ID NO. 16 does not express any protein of that size.

### III. Discussion

Search for Protein in Databases with the Composition of EAA Required by Humans: Analysis of Ratios and Length Distribution.

None of the 56,827,426 sequences annotated in PDB and Uniprot in a length range of 1-260 AA satisfied the requirements for each of the 9 EAA, having at most 4 EAA in the 1× ratio recommended in the human diet.

This was found to be due to the ratios in which AA were found in the annotated proteins and the very low probability of finding proteins of a certain length that would satisfy the requirements of each of the EAA. The AA required in the lowest amount in human daily intake as reported by the WHO were tryptophan, histidine and methionine. These EAA were found in lower ratios in the proteins of the databases analyzed (PDB, SwissProt and TrEMBL), although in ratios that were greater than the required 1× ratios. Thus, by increasing the required ratio of each EAA by 2-4 times more, proteins were found to satisfy the RDA of at least 8 EAA.

Length distributions showed that 45% of the sequences annotated in PDB and Uniprot have 1-250 AA. The search for proteins with the required EAA composition had been carried out using a maximum length of 260 AA because a longer protein would have a higher content of non-essential AA and more AA substitutions would have to be made to the protein to satisfy the EAA content.

The possible minimum length that the proteins should have to satisfy the requirement of each EAA, calculated by the formula:

$$P / n * 118.736 = V$$

was 560 AA for tryptophan. Because of this, it was highly unlikely that the code would find proteins of less than 260 AA that would satisfy the 9 EAA requirement, as the required W ratio was never satisfied in this length range. When using a length of 1000 AA, it was found that there were also no proteins with more than 4 EAA in the recommended 1× ratio due to the low probability of this happening. For example, for a sequence of at least 560 AA to have a tryptophan would occur only once in 19\hat{ }560 sequences (1/19\hat{ }560) and in a sample of 3-4.5×10\hat{ }5 sequences of 1-1000 AA this was very unlikely. However, finding proteins that satisfied the requirement of up to 8 EAA showed that proteins are not randomly present, even when their AA composition is very similar.
Ratios in which Each AA is Found at the Proteome Level.

A recent study entitled “The Distribution of Biomass on Earth” has become the first major estimate of the total biomass existing on our planet (Bar-On and Phillips, 2018). This research shows that dietary choices have a large effect on the habitats of living things, wherein 60% of mammals on the planet are livestock (cattle, sheep, goats and pigs) and 70% of birds are poultry. This led to think that the human diet could be inducing a trend in the AA composition of organisms with a higher content of EAA in the proteins of the organisms from which food production is based such as beef, pork, chicken, etc.

Using a bioinformatics approach made it possible to quickly analyze the ratios in which each of the 20 AA were found, not only in the whole database, as had been done previously, but also at the proteome level.

An interesting finding is that the composition of AA is conserved in plants, animals, bacteria, fungi and viruses. The 19 proteomes analyzed had very similar AA ratios. On average, the ratio of each AA deviated from the mean between 0.0057 and 0.0175.

Influenza type A, HIV and SARS-COV-2 viruses also followed the same pattern in AA composition (L, R and E in higher ratio and W, M and H in lower ratio).

It had previously been observed that the probability of finding proteins with the desired composition of certain EAA was very low, an example was the case of tryptophan, wherein the probability of finding a sequence of 560 AA having a W was 1/19\hat{ }559. However, having found results of proteins with 8 EAA in the recommended ratio showed that proteins are not random, even though their AA composition seems to be random (0.047 on average of each AA), since there are other influencing factors, such as the bioenergetic cost of the AA.

Akashi and Gojobori in 2001 published a paper in the scientific journal PNAS, showing that the composition of AA in Bacillus subtilis and E. coli was a reflection of natural selection for enhanced metabolic efficiency. The total metabolic cost of biosynthesis in E. coli was obtained by taking into account the number of phosphate bonds contained in ATP and GTP molecules, as well as the number of hydrogen atoms available in NADH, NADPH and FADH2 molecules, assuming 2 P per H.

Tryptophan (W) is the most expensive AA with 74.3 ATP molecules followed by phenylalanine (52), histidine (38.3), methionine (34.3), isoleucine (32.3), lysine (30.3), leucine (27.3), arginine (27.3), cysteine (24.7), valine (23.3), proline (20.3) and threonine (18.7). With a lower cost of biosynthesis are the AA glutamine (Q), glutamic acid (E), asparagine (N), aspartate (D), alanine (A), glycine (G) and serine(S).

As can be seen, the metabolism routes of the EAA are bioenergetically more expensive than those of the non-essential amino acids, which is consistent with the results obtained in this project, wherein the AA that were found in lower ratio were tryptophan, methionine and histidine. One of the AA found in lower ratio was tryptophan, which has the highest bioenergetic cost of the 20 AA. Leucine, arginine and glutamic acid were found in the highest ratio in the proteomes analyzed, which are of lower bioenergetic cost.

Search for Protein Mixtures with the Appropriate EAA Composition.

When searching for protein mixtures that satisfied the human requirement for EAA, it was found that the RDA of up to 4 EAA was satisfied.

If the sequences of more than 2 proteins were concatenated, the code no longer yielded positive results because having a mixture of proteins the EAA content would be diluted among many other non-essential AA. An example of this would be mixing 100 g of beef with 100 g of egg. Eggs contain 1.001 g of lysine (K), which satisfies 47.66% of the daily requirement of lysine (2100 mg); whereas, beef has 2.002 g lysine, which satisfies 95.33% of the requirement of this EAA. If these amounts are added together, it can be seen that the total amount ingested exceeds the total amount of lysine required. However, the ratio of lysine in that mixture is diluted.

The fact that humans have to consume a greater amount of protein results in an increased load of certain AA. It is well known that many amino acid residues of proteins are susceptible to oxidation by various reactive oxygen species (ROS) and that these oxidized proteins accumulate during aging. Methionine and cysteine residues in proteins are particularly sensitive to this oxidation, so consuming proteins with high amounts of these AA could be harmful to health (Stadtman et al., 2005).

An ideal protein, with the exact balance of AA would be of great relevance for the general public, as well as for susceptible population sectors due to the quality of proteins, such as the elderly and athletes. The protein intake of athletes should be higher to preserve muscle mass with a requirement of up to 2.4 g/kg. An athlete weighing 80 kg would need to consume about 192 g of protein per day. Natural sources of protein such as beef have about 22 g of protein per 100 g of meat, so to satisfy this protein requirement an athlete would need to consume about 872 g of protein sources daily. This would be detrimental to health due to the increased load of AA which has been shown to be a source of AGE, compounds that impulse proinflammatory and prooxidative nephrotoxicity (Goldberg et al., 2004; Uribarri et al., 2005). In people with hypertension, diabetes, or older adults with decreased kidney function, having to consume so much protein to satisfy the daily requirement of AA could also have deleterious effects with an increase in markers of kidney damage and an increased risk of developing cardiovascular disease (Wrone et al., 2003, Hoogeveen et al., 1998).

Search for Proteins that Complement Natural Protein Sources: Eggs, Milk, Beef, Fish, Pork and Chicken.

When searching for proteins that would complement the EAA content of natural protein sources such as beef, chicken, egg, milk, pork and fish, it was found that no sequence annotated in PDB, SwissProt or TrEMBL in a length range of 1-260 AA can complement the EAA content of these foods to satisfy the required 1× ratio of each EAA.

As observed in the search for proteins with the appropriate composition of AA, the requirement for a greater number of AA was satisfied as the ratio increased. At 2-4 times the required ratios for each of the 9 EAA, the best complementing natural sources were found to be egg with up to 5 EAA in the 2-3× and 3-4× ratios and milk with up to 7 EAA in the 3-4× ratio, 6 EAA in the 2-3× ratio and 3 EAA in the 1× ratio. In the case of the proteins that complemented milk with 6 or 7 EAA and egg with 5 EAA, it was analyzed whether they could serve as a food supplement for people with phenylketonuria; however, all of them had an excess of the AA phenylalanine.

When analyzing the EAA content in 100 g of these foods, it was observed that milk is less rich in EAA (1795 mg EAA/100 g) along with egg (6597 mg EAA/100 g); while beef (10,448 mg EAA/100 g) and chicken (11,858 mg EAA/100 g) have the highest EAA content. The fact that better results were found for proteins complementing milk and egg could be due to the fact that these foods have the lowest EAA content, which would make it easier to supplement a food that has almost no EAA in the required ratio, because the ratios in which the EAA are found exceed the required 1× ratios.

### Choice of the Protein Closest to the Optimal EAA Composition and Generation of Collagen Protein Variants.

A search of databases for proteins for human consumption led to the choice of collagen protein from Bos taurus. This choice was based on the previous results, wherein it was seen that no protein or mixture of proteins satisfies 1× the required ratios of each of the 9 EAA. Collagen protein was chosen because of its proximity to the daily requirement of EAA and the minimum number of AA substitutions to be made. Collagen fragments have been previously expressed in Pichia pastoris and it is a protein that is secreted into the extracellular medium, which is very useful in terms of purification because P. pastoris is a yeast that secretes few proteins (Cereghino et al., 2000).

A very relevant issue about collagen protein has to do with its null allergenicity. Previous immunoblotting studies have shown that collagen is not allergenic since it has no binding sites against human IgE antibodies (Wijaya et al., 2020; Hansen et al., 2004).

The collagen protein would require adding 2 lysines, 1 histidine, 2 valines and removing 2 threonines to satisfy the RDA of each EAA, which would involve carrying out 7 substitutions in an 86 AA protein. In several proteins, mutating a single AA residue has been shown to be counterproductive to protein structure and function (Purton et al., 2001). However, in this case, as it is a protein focused on human consumption the protein is not required to have function although the structure could be relevant for collagen protein secretion, since it has been seen that collagen proteins in random alpha-helix conformation can be secreted into the medium, while collagen proteins in triple helix have not been able to be secreted, but remain intracellularly (Nokelainen et al., 2001; Williams et al., 2008; He et al., 2015; Wang et al., 2014; Bin et al., 2011; Pakkanen et al., 2006).

In the collagen variants generated in silico, proline and glycine were excluded as positions of the protein wherein they could be mutated to avoid folding problems, since these residues are the ones that confer the helical structure to the protein. Nine variants were found to have 2 to 3 times the desired ratio of EAA, with a percentage difference from the original collagen protein of 13.9%.

Cloning of the Gene Coding for Bos taurus Collagen Protein into an Inducible and a Constitutive Expression Vector.

The P. pastoris system is proposed for collagen protein expression, as it is a GRAS (Generally Recognized as Safe) organism that has been approved for food product production by the Food and Drug Administration (FDA) (Ahmad et al., 2014).

Yields in the production of recombinant human collagen protein have been up to 14.8 g/L in P. pastoris (Werten et al., 1999) and it is a highly industrially used system that can be scaled up from a flask to high cell density cultures (Cereghino and Cregg, 2000). Induction of recombinant protein expression with methanol is the most commonly used model (Cereghino and Cregg, 2000). However, the proposal of the present invention consists of a glucose-inducible promoter, given the sustainable focus of the project and that it is a protein targeted for human consumption whose induction with methanol would be toxic (Prielhofer et al., 2018). On the other hand, genetic engineering has been used to modify plants, for example, corn, which have an increased amount of the amino acid lysine that they produce (https://www.ncbi.nlm.nih.gov/pmc/articles/PMC2442549/). However, this approach does not solve the problems of the excessive use of water required for its production, since it only provides one of the eleven essential amino acids and is produced in a plant whose proteins are very diluted and not very available to humans.

Currently, the intake of essential free amino acids is used to address the problem of sarcopenia, particularly leucine. (https://www.ncbi.nlm.nih.gov/pmc/articles/PMC3183816/).

Finally, it is important to point out that there is no protein in nature that provides humans with the minimum amount of essential amino acids required in their daily diet. For the purposes of the present invention, by minimum amount it should be understood that if the human being requires to ingest 20 grams of essential amino acids, with the natural sources in order to obtain that amount he must consume 5 or more times that amount in grams of the natural sources, the minimum would be 20 grams. This explains the need to ingest large quantities of food, which has a negative impact on the environment.

On the other hand, there are proteins that come close to containing the ratio of essential amino acids that can be used to change their essential amino acid composition with a minimum number of changes and achieve an optimized protein in composition and amount ingestible for human nutrition.

Although exemplary embodiments of the present invention have been described with reference to the drawings, it should be understood that these exemplary embodiments are merely illustrative and are not intended to limit the scope of the present invention. It is likely that a person skilled in the art could make various changes and modifications to said embodiments, but without departing from the true scope and spirit of said present invention, wherein said changes and modifications must be intended to be included in the scope of the present invention as drafted in the appended claims, as may be the case of using the algorithmic method developed by the inventors of the present invention, which, besides allowing to obtain the optimized protein comprising all the amino acids essential in human nutrition, allows to obtain any other proteins with different ratios of amino acids and to be used in any other living being.

In the particularly preferred embodiment described herein, it should be understood that the optimized protein comprising all essential amino acids in human nutrition can be implemented in other ways, whereby said embodiment of the method is merely exemplary.

The person skilled in the art may understand that, in addition to the mutual exclusion of characteristics, any possible combination may be adopted to incorporate and combine all characteristics disclosed by the description (including the appended claims, abstract and drawings) and all processes or units of any method disclosed as such. Unless expressly stated otherwise, each characteristic disclosed by the present description (including the appended claims, abstract and drawings) may be replaced by an alternative characteristic providing the same, equivalent or similar purpose.

Furthermore, the person skilled in the art may understand that, even though some embodiments described herein comprise some characteristics included in other embodiments, rather than other characteristics, combinations of characteristics of different embodiments are considered to fall within the scope of the present invention and form different embodiments. For example, in the claims, wherein any of the embodiments for which protection is sought can be used in various combination modes.

References in the description to “an embodiment” or “embodiments” indicate that the embodiment described may include a particular aspect, feature, structure or characteristic, but not all embodiments necessarily include such aspect, feature, structure or characteristic. In addition, such phrases may, but need not necessarily, refer to the same embodiment mentioned elsewhere in the specification. In addition, when describing a particular aspect, feature, structure or characteristic in relation to an embodiment, it is within the knowledge of the person skilled in the art to affect or connect that aspect, feature, structure or characteristic with other embodiments, whether or not they have been explicitly described. In other words, any element or characteristic may be combined with any other element or feature in different embodiments, unless there is an obvious or inherent incompatibility, or it is specifically excluded.

As such, an invention is disclosed in terms of preferred embodiments thereof that comply with each and every object of the present invention, as set forth above and provides an optimized protein comprising all essential amino acids in ratios suitable for human nutrition. Of course, the person skilled in the art may contemplate various changes, modifications and alterations to the teachings of the present invention, but without departing from the intended spirit and scope thereof. It is intended that the present invention be limited only by the terms of the appended claims.

The terminology used herein is intended only to describe particular or preferred embodiments and is not intended to limit the invention. As described herein, the singular forms “a”, “an” and “the” are intended to include the plural forms as well, unless the context clearly indicates otherwise. It shall be further understood that the terms “comprises” and/or “comprising”, when described in this specification, specify the presence of stated characteristics, integers, stages, operations, elements, and/or components, but do not exclude the presence or addition of one or more characteristics, integers, stages, operations, elements, components, and/or groups thereof. As described herein, the term “and/or” includes any and all combinations of any one or more of the listed associated elements. Throughout the description, unless explicitly described otherwise, the word “comprise” and variations such as “comprises” or “comprising” will be understood to imply the inclusion of the stated elements, but not the exclusion of any other elements.

The claims may be drafted to exclude any optional elements. As such, this statement is intended to serve as a background basis for the use of exclusive terminology, such as “solely”, “only”, “only” and the like, in connection with the mention of claim elements or the use of a “negative” limitation. The terms “preferably”, “preferred”, “prefer”, “optionally”, “may” and similar terms are used to indicate that an element, condition or stage referred to is an optional (not required) feature of the invention.

In summary, even though in the foregoing detailed description of the present invention reference has been made to certain embodiments of the optimized protein comprising all the essential amino acids in the ratios suitable for human nutrition, it should be emphasized that numerous modifications to said embodiments are possible, but without departing from the true scope of the present invention, in such a way that the characteristics described in the aforementioned embodiments, shown in the figures and claimed in the claiming chapter, as well as the characteristics of different embodiments which have not been described herein, may be used individually or in any arbitrary combination for the realization of said present invention. Accordingly, it should be understood that the embodiments of the present invention are illustrative only and are not intended to limit the scope of the present invention except as set forth in the prior art and the appended claims.

1. A protein optimized for human nutrition, characterized in that it comprises one or more amino acid sequences selected from the group of sequences: SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; SEQ ID NO. 14; SEQ ID NO. 15; SEQ ID NO. 16; SEQ ID NO. 17; SEQ ID NO. 18; SEQ ID NO. 19; SEQ ID NO. 20, either individually or in combinations of two or more, wherein said optimized protein comprises all essential amino acids in ratios suitable for human nutrition.

2. The protein optimized for human nutrition according to claim 1, further characterized in that it comprises the amino acid sequences as set forth in SEQ ID NO. 12 and SEQ ID NO. 16, wherein said optimized protein comprises all essential amino acids in ratios suitable for human nutrition.

3. The protein optimized for human nutrition according to claim 1, further characterized in that it comprises the amino acid sequence as set forth in SEQ ID NO. 16, wherein said optimized protein comprises all essential amino acids in ratios suitable for human nutrition.

4. The protein optimized for human nutrition according to claim 3, further characterized in that the corresponding nucleotide sequence for protein SEQ ID NO. 16 is as follows:
5′-
CAACCACCCAGGTCCACCAGGTCCACCAGGTCCACCAGTTTCTGCTATG
TTGCCAGGTCCATTCGGTTTGCCAGGTTTCCCAGGTACTCCAGGTATGA
AGGGTATTCAAGGTGAGAGAGGTTTGCCAGGTGAGAAGGGTGAGGTTGG
TTTGCCAGGTCCACCAGGTCCACAAGGTGAGTCTAGATTGGGTCCACCA
GGTTCTACTGGTTCTAGAGGTGTTCCAGGTCCACCAGGTAGACCAGGTG
ACTCTGGTATTAAG-3′

5. The protein optimized for human nutrition according to claim 4, further characterized in that the amino acid sequence SEQ ID NO. 16 is expressed in yeast Pichia pastoris under 2 different promoters: pGAP and pAOX1; wherein pGAP is a system in which gene and protein expression is induced in the presence of glucose and pAOX1 in the presence of methanol.

6. A method for selecting a protein sequence for human nutrition that comes closest to containing the appropriate ratio of essential amino acids, characterized in that it comprises performing a search of public databases of protein sequences comprising the following steps: 1) defining the ratio of essential amino acids per gram of total protein desired to be found in any protein (PAAE), wherein this ratio is specified in a range of values (RVPAAE); 2) defining how many essential amino acids must comply with the RVPAAE (AAEE: Expected Essential Amino Acids), which can be from 1 to 9; 3) searching in protein sequence databases, those that satisfy steps 1) and 2); 4) repeating steps 1), 2) and 3) for different possible values of AAEE; 5) selecting the proteins whose AAEE are higher, preferably those proteins that satisfy a greater number of essential amino acids in the adequate ratio for human nutrition; where, from the sequence selected in the previous procedure, changes are made in that sequence to bring it to contain the essential amino acids in the desired ratios of essential amino acids.

7. A method for synthesizing a protein optimized for human nutrition, characterized in that it comprises: a) performing a search in public databases of protein sequences; b) starting the power of the system; c) calculating the value of the energy function of the sequence; d) printing the solution, provided that the value of the energy function of the sequence is equal to 0; otherwise continue with the following steps; e) randomly selecting the positions in the sequence to be mutated; the number of positions is the method parameter specified in the following step f); f) verifying that the selected positions are among the positions susceptible to be changed specified by the parameter of the corresponding method with in step c); g) randomly selecting the essential amino acids for which the amino acids present in the selected positions are to be changed; h) carrying out the substitutions specified in step f) on the positions selected in step e); if the new sequence reduces the energy value, keep it, otherwise evaluate whether to keep that sequence by applying the following formula:
$${e}^{( previous energy - new energy ) / temperature}$$
if the result gives a number greater than a randomly chosen number between 0 and 1, then the sequence is retained for the next cycle; i) repeating the above steps until the number of sequences printed equals the method parameter specified in step h), or a value in the system energy less than 1 has been reached; and j) synthesizing or producing the protein with the desired amino acid sequence(s).

8. The method for synthesizing a protein optimized for human nutrition according to claim 7, characterized in that the production or synthesis of the protein comprises the steps of: expressing the protein in yeast Pichia pastoris under 2 different promoters: pGAP and pAOX1; wherein pGAP is a system in which gene and protein expression is induced in the presence of glucose and pAOX1 in the presence of methanol and wherein the corresponding nucleotide sequence for protein of SEQ ID NO. 16 is as follows:
5′-
CAACCACCCAGGTCCACCAGGTCCACCAGGTCCACCAGTTTCTGCTATG
TTGCCAGGTCCATTCGGTTTGCCAGGTTTCCCAGGTACTCCAGGTATGA
AGGGTATTCAAGGTGAGAGAGGTTTGCCAGGTGAGAAGGGTGAGGTTGG
TTTGCCAGGTCCACCAGGTCCACAAGGTGAGTCTAGATTGGGTCCACCA
GGTTCTACTGGTTCTAGAGGTGTTCCAGGTCCACCAGGTAGACCAGGTG
ACTCTGGTATTAAG-3′

9. The method according to claim 7, characterized in that the searching public protein sequence databases comprises the following steps: 1) defining the ratio of essential amino acids per gram of total protein desired to be found in any protein (PAAE), wherein this ratio is specified in a range of values (RVPAAE); 2) defining how many essential amino acids must comply with the RVPAAE (AAEE: Expected Essential Amino Acids), which can be from 1 to 9; 3) searching in databases of protein sequences, those that satisfy the steps 1) and 2); 4) repeating steps 1), 2) and 3) for different possible values of AAEE; 5) select the proteins whose AAEE are higher, preferably those proteins that satisfy a greater number of essential amino acids in the adequate ratio for human nutrition; wherein additionally, from the sequence selected in the previous procedure, changes are made to the sequence to bring it to contain the essential amino acids in the desired ratios of essential amino acids.

10. A food formulation or supplement characterized in that it comprises the protein optimized for human nutrition according to claim 1, in combination with acceptable vehicles and/or excipients.

11. The formulation or food supplement, according to claim 10, for use in the treatment of a nutritional deficit.
